SEQUENCE LISTING (48 sequences)

SEQ ID NO: 1 - protein, 478 aa, Artificial
  anti-MUC1 scFvFc (AG SEED)
MQMQLVQSEAELKKPGASVKVSCKASGYSFTSHFMHWVRQAPGQGLEWMGWIDPVTGGTKYAQNFQGWVTMTRDTSIRTAYLELSRLRSDDTAMYYCAREARADRGQFDKWGQGTLVTVASGGGGSGGGGSGGGGSQSVLTQPPSVSVAPGKTARITCGGNNIGSKSVHWYQQKPGQAPALVIYYGSNRPSGIPERFSGSNSGNTATLTISRVEAGDEADYYCQVWDSSSDWVFGGGTKLTVLKPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 2 - protein, 453 aa, Artificial
  anti-EGFR Fab(heavy)Fc (GA SEED)
MEVQLVQSGAEVKKPGASVKVSCKASGFSLTNYGVHWMRQAPGQGLEWIGVIWSGGNTDYNTPFTSRVTITSDKSTSTAYMELSSLRSEDTAVYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 3 - protein, 215 aa, Artificial
  anti-EGFR Fab(light chain)
MDIQMTQSPSSLSASVGDRVTITCRASQSIGTNIHWYQQKPGKAPKLLIKYASESISGVPSRFSGSGYGTDFTLTISSLQPEDVATYYCQQNNNWPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 10 aa, Artificial
  CDRH1 motif for HT186-D11 and 1993 series antibodies
  VARIANT (3)..(3): Xaa is Ser or Pro
  VARIANT (5)..(5): Xaa is Thr or Asn
  VARIANT (6)..(6): Xaa is Gly, Asp, or Ser
  VARIANT (7)..(7): Xaa is His or Asn
  VARIANT (8)..(8): Xaa is Tyr or Phe
GYXFXXXXMH

SEQ ID NO: 5 - protein, 17 aa, Artificial
  CDRH2 motif for HT186-D11 and 1993 series antibodies
  VARIANT (8)..(8): Xaa is Gly or Glu
  VARIANT (10)..(10): Xaa is Lys or Arg
  VARIANT (14)..(14): Xaa is Asn or Asp
WIDPVTGXTXYAQXFQG

SEQ ID NO: 6 - protein, 11 aa, Artificial
  CDRH3 motif for HT186-D11 and 1993 series antibodies
  VARIANT (2)..(2): Xaa is Val or Ala
  VARIANT (3)..(3): Xaa is Thr or Arg
  VARIANT (4)..(4): Xaa is Gly or Ala
  VARIANT (5)..(5): Xaa is Asp or Ser
EXXXXRGQFDK

SEQ ID NO: 7 - protein, 11 aa, Artificial
  CDRL1 for HT186-D11 and 1993 series antibodies
GGNNIGSKSVH

SEQ ID NO: 8 - protein, 7 aa, Artificial
  CDRL2 for HT186-D11 and 1993 series antibodies
YGSNRPS

SEQ ID NO: 9 - protein, 10 aa, Artificial
  CDRL3 for HT186-D11 and 1993 series antibodies
QVWDSSSDWV

SEQ ID NO: 10 - protein, 16 aa, Artificial
  VNTR peptide of MUC1
APDTRPAPGSTAPPAC

SEQ ID NO: 11 - protein, 478 aa, Artificial
  anti-MUC1 scFvFc (AG SEED) with non-natural amino acids (e.g., pAMF) introduced at Xaa sites
  MUTAGEN (269)..(269): Non-natural amino acid, e.g., para azidomethyl L-phenylalanine (pAMF)
  MUTAGEN (435)..(435): Non-natural amino acid, e.g., para azidomethyl L-phenylalanine (pAMF)
MQMQLVQSEAELKKPGASVKVSCKASGYSFTSHFMHWVRQAPGQGLEWMGWIDPVTGGTKYAQNFQGWVTMTRDTSIRTAYLELSRLRSDDTAMYYCAREARADRGQFDKWGQGTLVTVASGGGGSGGGGSGGGGSQSVLTQPPSVSVAPGKTARITCGGNNIGSKSVHWYQQKPGQAPALVIYYGSNRPSGIPERFSGSNSGNTATLTISRVEAGDEADYYCQVWDSSSDWVFGGGTKLTVLKPKSSDKTHTCPPCPAPELLGGPSVXLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTXAVTSKLTVDKSRWQQGNVFSCSVMHEALHNHYTQKTISLSPGK

SEQ ID NO: 12 - protein, 453 aa, Artificial
  anti-EGFR Fab(heavy)Fc (GA SEED) with non-natural amino acids (e.g., pAMF) introduced at Xaa sites
  MUTAGEN (183)..(183): Xaa is an unnatural amino acid, e.g., para azidomethyl L-phenylalanine (pAMF)
  MUTAGEN (244)..(244): Xaa is an unnatural amino acid, e.g., para azidomethyl L-phenylalanine (pAMF)
MEVQLVQSGAEVKKPGASVKVSCKASGFSLTNYGVHWMRQAPGQGLEWIGVIWSGGNTDYNTPFTSRVTITSDKSTSTAYMELSSLRSEDTAVYYCARALTYYDYEFAYWGQGTLVTVSSASTKGPSVFPLAPSSKSTSGGTAALGCLVKDYFPEPVTVSWNSGALTSGVHTFPAVLQSSGLXSLSSVVTVPSSSLGTQTYICNVNHKPSNTKVDKRVEPKSCDKTHTCPPCPAPELLGGPSVXLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 13 - protein, 8 aa, Artificial
  CDRH1 for hC225
GFSLTNYG

SEQ ID NO: 14 - protein, 7 aa, Artificial
  CDRH2 for hC225
IWSGGNT

SEQ ID NO: 15 - protein, 13 aa, Artificial
  CDRH3 for hC225
ARALTYYDYEFAY

SEQ ID NO: 16 - protein, 6 aa, Artificial
  CDRL1 for hC225
QSIGTN

SEQ ID NO: 17 - protein, 4 aa, Artificial
  CDRL2 for hC225
YASE

SEQ ID NO: 18 - protein, 9 aa, Artificial
  CDRL3 for hC225
QQNNNWPTT

SEQ ID NO: 19 - protein, 98 aa, Artificial
  CH1 within hC225 heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 20 - protein, 98 aa, Artificial
  CH1 within hC225 heavy chain with non-natural amino acid (e.g., pAMF) introduced at Xaa
  MUTAGEN (63)..(63): Xaa is a non-natural amino acid, e.g., para azidomethyl L-phenylalanine (pAMF)
ASTKGPSVFPLAPSSKSTSGGTAALGCLVKDYFPEPVTVSWNSGALTSGVHTFPAVLQSSGLXSLSSVVTVPSSSLGTQTYICNVNHKPSNTKVDKRV

SEQ ID NO: 21 - protein, 107 aa, Artificial [sequence omitted]

SEQ ID NO: 22 - protein, 40 aa, Artificial
  peptide from human MUC1
APDTRPAPGSTAPPAHGVTSAPDTRPAPGSTAPPAHGVTS

SEQ ID NO: 23 - protein, 15 aa, Artificial
  human MUC1 truncated peptide
APDTRPAPGSTAPPA

SEQ ID NO: 24 - protein, 15 aa, Artificial
  human MUC1 truncated peptide
PAPGSTAPPAHGVTS

SEQ ID NO: 25 - protein, 15 aa, Artificial
  human MUC1 truncated peptide
TAPPAHGVTSAPDTR

SEQ ID NO: 26 - protein, 15 aa, Artificial
  human MUC1 truncated peptide
HGVTSAPDTRPAPGS

SEQ ID NO: 27 - protein, 5 aa, Artificial
  human MUC1 minimal consensus motif
TRPAP

SEQ ID NO: 28 - protein, 6 aa, Artificial
  human MUC1 minimal consensus motif
DTRPAP

SEQ ID NO: 29 - protein, 5 aa, Artificial
  CDRH1 for HT186-D11 - Kabat
GHYMH

SEQ ID NO: 30 - protein, 17 aa, Artificial
  CDRH2 for HT186-D11 - Kabat
WIDPVTGGTKYAQNFQG

SEQ ID NO: 31 - protein, 11 aa, Artificial
  CDRH3 for HT186-D11 - Kabat
EVTGDRGQFDK

SEQ ID NO: 32 - protein, 7 aa, Artificial
  CDRH1 for HT186-D11 - Chothia
GYSFTGH

SEQ ID NO: 33 - protein, 6 aa, Artificial
  CDRH2 for HT186-D11 - Chothia
DPVTGG

SEQ ID NO: 34 - protein, 11 aa, Artificial
  CDRH3 for HT186-D11 - Chothia
EVTGDRGQFDK

SEQ ID NO: 35 - protein, 5 aa, Artificial
  CDRH1 for 1993-H02 - Kabat
SHFMH

SEQ ID NO: 36 - protein, 17 aa, Artificial
  CDRH2 for 1993-H02 - Kabat
WIDPVTGGTKYAQNFQG

SEQ ID NO: 37 - protein, 11 aa, Artificial
  CDRH3 for 1993-H02 - Kabat
EARADRGQFDK

SEQ ID NO: 38 - protein, 7 aa, Artificial
  CDRH1 for 1993-H02 - Chothia
GYSFTSH

SEQ ID NO: 39 - protein, 6 aa, Artificial
  CDRH2 for 1993-H02 - Chothia
DPVTGG

SEQ ID NO: 40 - protein, 11 aa, Artificial
  CDRH3 for 1993-H02 - Chothia
EARADRGQFDK

SEQ ID NO: 41 - protein, 120 aa, Artificial
  VH for HT186-D11
QMQLVQSEAELKKPGASVKVSCKASGYSFTGHYMHWVRQAPGQGLEWMGWIDPVTGGTKYAQNFQGWVTMTRDTSIRTAYLELSRLRSDDTAMYYCAREVTGDRGQFDKWGQGTLVTVAS

SEQ ID NO: 42 - protein, 120 aa, Artificial
  VH for 1993-H02
QMQLVQSEAELKKPGASVKVSCKASGYSFTSHFMHWVRQAPGQGLEWMGWIDPVTGGTKYAQNFQGWVTMTRDTSIRTAYLELSRLRSDDTAMYYCAREARADRGQFDKWGQGTLVTVAS

SEQ ID NO: 43 - protein, 107 aa, Artificial
  VL for HT186-D11 and for 1993-H02
QSVLTQPPSVSVAPGKTARITCGGNNIGSKSVHWYQQKPGQAPALVIYYGSNRPSGIPERFSGSNSGNTATLTISRVEAGDEADYYCQVWDSSSDWVFGGGTKLTVL

SEQ ID NO: 44 - protein, 120 aa, Artificial
  VH for 1993-E03
QMQLVQSEAELKKPGASVKVSCKASGYSFNDHFMHWVRQAPGQGLEWMGWIDPVTGGTKYAQNFQGWVTMTRDTSIRTAYLELSRLRSDDTAMYYCAREARADRGQFDKWGQGTLVTVAS

SEQ ID NO: 45 - protein, 120 aa, Artificial
  VH for 1993-H08
QMQLVQSEAELKKPGASVKVSCKASGYSFTGHYMHWVRQAPGQGLEWMGWIDPVTGETKYAQNFQGWVTMTRDTSIRTAYLELSRLRSDDTAMYYCAREARADRGQFDKWGQGTLVTVAS

SEQ ID NO: 46 - protein, 120 aa, Artificial
  VH for 1993-G09
QMQLVQSEAELKKPGASVKVSCKASGYSFTDHYMHWVRQAPGQGLEWMGWIDPVTGETKYAQDFQGWVTMTRDTSIRTAYLELSRLRSDDTAMYYCAREARADRGQFDKWGQGTLVTVAS

SEQ ID NO: 47 - protein, 120 aa, Artificial
  VH for 1993-E04
QMQLVQSEAELKKPGASVKVSCKASGYSFTGNYMHWVRQAPGQGPEWMGWIDPVTGETKYAQNFQGWVTMTRDTSIRTAYLELSRLRSDDTAMYYCAREARASRGQFDKWGQGTLVTVAS

SEQ ID NO: 48 - protein, 120 aa, Artificial
  VH for 1993-D01
QMQLVQSEAELKKPGASVKVSCKASGYPFTGHYMHWVRQAPGQGPEWMGWIDPVTGETRYAQNFQGWVTMTRDTSIRTAYLELSRLRSDDTAMYYCAREARADRGQFDKWGQGTLVTVAS

(19) United States
(12) Patent Application Publication
(10) Pub. No.: US 2023/0310629 A1
(43) Pub. Date: Oct. 5, 2023

(54) BISPECIFIC ANTIBODY-DRUG CONJUGATES TARGETING EGFR AND MUC1 AND USES THEREOF

(71) Applicant: Merck Patent GmbH, Darmstadt (DE)

(72) Inventors: Christine Knuehl, Darmstadt (DE); Lars Toleikis, Kleinniedesheim (DE); Christiane Amendt, Mühltal (DE); Achim Doerner, Darmstadt (DE); Alice Yam, Belmont, CA (US); Xiaofan Li, Belmont, CA (US); Ryan Stafford, Foster City, CA (US); Robert Henningsen, Redwood City, CA (US); Sihong Zhou, San Mateo, CA (US)

(21) Appl. No.: 18/007,680

(22) PCT Filed: Jun. 3, 2021
(86) PCT No.: PCT/US2021/035600

§ 371 Date: Dec. 1, 2022

Related U.S. Application Data
(60) Provisional application No. 63/034,296, filed on Jun. 3, 2020.

(51) Int. Cl. A61K 47/68; C07K 16/30; C07K 16/28; A61P 35/00

(52) U.S. Cl. CPC A61K 47/6811; A61K 47/6849; C07K 16/3092; C07K 16/2863; A61P 35/00; A61K 47/6869; A61K 47/6857; A61K 47/6851; A61K 47/6865; A61K 2039/505

(57) ABSTRACT

Provided are immunoconjugates comprising bispecific anti-MUC 1/EGFR antibodies conjugated to hemiasterlin-based moieties via cleavable linkers, and pharmaceutical compositions thereof. Provided also are methods of treating cancer using such immunoconjugates and pharmaceutical compositions.

### CROSS-REFERENCE TO RELATED APPLICATIONS

The present application claims the benefit of and priority to U.S. Provisional Patent Application No. 63/034,296, filed Jun. 3, 2020, the entire content of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Jun. 1, 2021, is named EMD-016WO_Sequence_Listing.txt and is 38 kilobytes in size.

### FIELD OF THE INVENTION

The field of the invention is molecular biology, immunology, and oncology. More particularly, the field is therapeutic antibody-drug conjugates.

### BACKGROUND

Epidermal growth factor receptor (EGFR; also known as ErbB1) is a transmembrane protein that is overexpressed in several epithelial cancers. Some EGFR mutations, including deletion mutations, point mutations, insertion mutations, and gene amplifications have been associated with cancer. Some EGFR mutations, as well as EGFR overexpression, are associated with poor prognosis and/or resistance to targeted EGFR inhibitors and other receptor tyrosine kinase inhibitors. Several novel pathways leading to escape from anti-EGFR therapy have recently been reported, highlighting the challenges of anti-EGFR therapy.

Additionally, EGFR is basally expressed in normal tissues throughout the body. Therefore, antibody therapies targeting EGFR may result in undesired off-target effects and enhanced toxicity.

Despite the efforts made to date, there remains a need for improved anti-cancer therapies.

### SUMMARY

The present disclosure provides novel bispecific antibody-drug conjugates that address both the lack of efficacy and the lack of tumor selectivity observed with some anti-EGFR therapeutics.

In one aspect, provided are immunoconjugates that comprise: (a) a bispecific antibody that binds to EGFR and MUC1 and (b) a plurality of hemiasterlin moieties. The bispecific antibody comprises: (i) a first polypeptide comprising a first engineered Fc domain and a single-chain Fv (scFv), wherein the scFv binds to MUC1, (ii) a second polypeptide comprising a second engineered Fc domain and a heavy chain of an Fab fragment, and (iii) a third polypeptide comprising a light chain of the Fab fragment; wherein the second and third polypeptide chains together define an Fab fragment that binds EGFR. The first polypeptide and the second polypeptide are covalently linked by one or more disulfide bonds formed between the first engineered Fc domain and the second engineered Fc domain. The second polypeptide and the third polypeptide are covalently linked by one or more disulfide bonds formed between the heavy chain of the second polypeptide and the light chain of the third polypeptide. The immunoconjugates also comprise (b) a plurality of hemiasterlin moieties, e.g., four hemiasterlin moieties. The first polypeptide and the second polypeptides each comprise at least one non-natural amino acid residue, and each hemiasterlin moiety is independently conjugated via a linker to one of the non-natural amino acid residues of the first polypeptide or the second polypeptide.

In certain embodiments, the first engineered Fc domain is different from the second engineered Fc domain. For example, the first and second engineered Fc domains each comprise strand-exchange engineered domains, which may, for example, comprise alternating segments of human IgA and IgG constant heavy chain-3 (C_{H}3) sequences.

In certain embodiments, the first engineered Fc domain comprises two non-natural amino acid residues, for example, at heavy chain positions F241 and F404 according to the EU index. In some embodiments, the first engineered Fc domain comprises no more than two non-natural amino acid residues.

In certain embodiments, the second engineered Fc domain comprises a non-natural amino acid residue, for example, at heavy chain position F241 according to the EU index. In some embodiments, the second engineered Fc domain comprises no more than one non-natural amino acid residue.

In certain embodiments, the Fab fragment comprises a non-natural amino acid residue. In some embodiments, the heavy chain of the Fab fragment within the second polypeptide comprises a non-natural residue, for example, at heavy chain position Y180 according to the EU index. In some embodiments, the Fab fragment comprises no more than one non-natural amino acid residue. In some embodiments, the heavy chain of the Fab fragment within the second polypeptide comprises a non-natural amino acid residue at heavy chain position Y180 according to the EU index.

In certain embodiments, each of the at least one non-natural amino acid residues is selected from the group consisting of p-acetyl-L-phenylalanine, O-methyl-L-tyrosine, 3-methyl-phenylalanine, O-4-allyl-L-tyrosine, 4-propyl-L-tyrosine, fluorinated phenylalanine, isopropyl-L-phenylalanine, p-azido-L-phenylalanine, p-acyl-L-phenylalanine, p-benzoyl-L-phenylalanine, p-iodophenylalanine, p-bromophenylalanine, p-amino-L-phenylalanine, isopropyl-L-phenylalanine, p-propargyloxyphenylalanine, and p-azidomethyl-L-phenylalanine. In certain embodiments, each of the at least one non-natural amino acid residues is para-azidomethyl-L-phenylalanine (pAMF).

In certain embodiments, the bispecific antibody is aglycosylated.

In certain embodiments, the first polypeptide comprises complementarity-determining regions (CDRs):
CDR-L1 comprising the amino acid sequence set forth in SEQ ID NO:7;
CDR-L2 comprising the amino acid sequence set forth in SEQ ID NO:8; and
CDR-L3 comprising the amino acid sequence set forth in SEQ ID NO:9.

In certain embodiments, the first polypeptide comprises complementarity-determining regions (CDRs):
CDR-H1 comprising the amino acid sequence set forth in SEQ ID NO:4,
CDR-H2 comprising the amino acid sequence set forth in SEQ ID NO:5, and
CDR-H3 comprising the amino acid sequence set forth in SEQ ID NO:6.

In certain embodiments, the first polypeptide comprises complementarity-determining regions (CDRs):
(a) (i) CDR-H1 comprising the amino acid sequence set forth in SEQ ID NO:29,
   (ii) CDR-H2 comprising the amino acid sequence set forth in SEQ ID NO:30, and
   (iii) CDR-H3 comprising the amino acid sequence set forth in SEQ ID NO:31; or
(b) (i) CDR-H1 comprising the amino acid sequence set forth in SEQ ID NO:32,
   (ii) CDR-H2 comprising the amino acid sequence set forth in SEQ ID NO:33, and
   (iii) CDR-H3 comprising the amino acid sequence set forth in SEQ ID NO:34.

In certain embodiments, the first polypeptide comprises: (a) a heavy chain variable (VH) region comprising the amino acid sequence set forth in SEQ ID NO:41; and (b) a light chain variable (VL) region comprising the amino acid sequence set forth in SEQ ID NO:43.

In certain embodiments, the first polypeptide comprises complementarity-determining regions (CDRs):
(a) (i) CDR-H1 comprising the amino acid sequence set forth in SEQ ID NO:35,
   (ii) CDR-H2 comprising the amino acid sequence set forth in SEQ ID NO:36, and
   (iii) CDR-H3 comprising the amino acid sequence set forth in SEQ ID NO:37; or
(b) (i) CDR-H1 comprising the amino acid sequence set forth in SEQ ID NO:38,
   (ii) CDR-H2 comprising the amino acid sequence set forth in SEQ ID NO:39, and
   (iii) CDR-H3 comprising the amino acid sequence set forth in SEQ ID NO:40.

In certain embodiments, the first polypeptide comprises:
(a) a heavy chain variable (VH) region comprising the amino acid sequence set forth in SEQ ID NO:42; and (b) a light chain variable (VL) region comprising the amino acid sequence set forth in SEQ ID NO:43.

In certain embodiments, the second polypeptide comprises complementarity-determining regions (CDRs):
CDR-H1 comprising the amino acid sequence set forth in SEQ ID NO:13,
CDR-H2 comprising the amin acid sequence set forth in SEQ ID NO:14, and
CDR-H3 comprising the amino acid sequence set forth in SEQ ID NO:15,

In certain embodiments, the third polypeptide comprises complementarity-determining regions (CDRs):
CDR-L1 comprising the amino acid sequence set forth in SEQ ID NO:16,
CDR-L2 comprising the amino acid sequence set forth in SEQ ID NO:17, and
CDR-L3 comprising the amino acid sequence set forth in SEQ ID NO:18.

In some embodiments, the first polypeptide has an amino acid sequence at least 99% identical to that set forth in SEQ ID NO:1. In some embodiments, the first polypeptide has an amino acid sequence as set forth in SEQ ID NO:11. In some embodiments, the second polypeptide has an amino acid sequence at least 99% identical to that set forth in SEQ ID NO:2. In some embodiments, the second polypeptide has an amino acid sequence as set forth in SEQ ID NO:12. In some embodiments, the third polypeptide has an amino acid sequence at least 99% identical to that set forth in SEQ ID NO:3. In some embodiments, the third polypeptide has an amino acid sequence as set forth in SEQ ID NO:3.

In certain embodiments, the linker is a cleavable linker, for example, valine-citrulline-p-aminobenzylalcohol (PABA).

In certain embodiments, the hemiasterlin moiety is a hemiasterlin derivative, for example, 3-aminophenyl-hemiasterlin.

In certain embodiments, the immunoconjugate comprises the following structure:

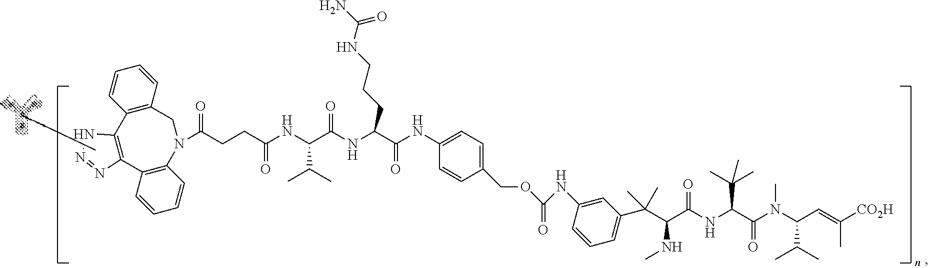

wherein n is 4.

In certain embodiments, provided are immunoconjugates comprising:
(a) a bispecific antibody that binds to EGFR and MUC1, the bispecific antibody comprising:
   (i) a first polypeptide comprising a first engineered Fe domain and a single-chain Fv fragment (scFv), wherein the scFv binds to MUC1, the first polypeptide chain comprising the amino acid sequence of SEQ ID NO:11 that comprises a non-natural amino acid residue at heavy chain positions F241 and F404 according to the EU index,
   (ii) a second polypeptide comprising a second engineered Fc domain and a heavy chain of an Fab fragment, the second polypeptide comprising the amino acid sequence of SEQ ID NO:12 that comprises a non-natural amino acid residue at positions Y180 and F241 according to the EU index, and
   (iii) a third polypeptide comprising a light chain of the Fab fragment, the third polypeptide comprising the amino acid sequence of SEQ ID NO:3;
   wherein the second and third polypeptide chains together define an Fab fragment that binds EGFR,
   wherein the first polypeptide and the second polypeptide are covalently linked by one or more disulfide bonds formed between the first engineered Fc domain and the second engineered Fc domain, and
   wherein the second polypeptide and the third polypeptide are covalently linked by one or more disulfide bonds formed between the heavy chain of the second polypeptide and the light chain of the third polypeptide; and
(b) a plurality of 3-aminophenyl hemiasterlin moieties, each independently conjugated via a cleavable valine-citrulline-p-aminobenzylalcohol linker to one of the non-natural amino acid residues.

In certain embodiments, the immunoconjugate comprises four 3-aminophenyl hemiasterlin moieties. In certain embodiments, each non-natural amino acid is para-azidomethyl-L-phenylalanine (pAMF).

In another aspect, provided are pharmaceutical compositions comprising an immunoconjugate as disclosed herein and a pharmaceutically acceptable carrier.

In another aspect, provided are methods of treating cancer comprising the step of: administering a therapeutically effective amount of an immunoconjugate or a pharmaceutical composition disclosed herein to a mammalian subject in need thereof, for example, a human mammalian subject and/or a subject diagnosed as having cancer.

In certain embodiments, the cancer comprises a solid tumor. For example, the cancer may be selected from the group consisting of breast cancer, lung cancer, esophageal cancer, head and neck cancer, cervical cancer, ovarian cancer, and gastric cancer. In some embodiments, the cancer is breast cancer, for example, triple negative breast cancer. In some embodiments, the cancer is lung cancer, for example, a non-small cell lung cancer (NSCLC), such as an NSCLC comprising an adenocarcinoma and/or a squamous cell carcinoma. In some embodiments, the cancer is esophageal cancer, for example, squamous esophageal cancer. In some embodiments, the cancer is head and neck cancer, for example, head and neck squamous cell carcinoma. In some embodiments, the cancer is cervical cancer. In some embodiments, the cancer is ovarian cancer. In some embodiments, the cancer is gastric cancer. In some embodiments, the cancer is mesothelioma. In some embodiments, the solid tumor is metastatic.

In certain embodiments, the cancer comprises a non-solid tumor, for example, multiple myeloma.

In certain embodiments, the cancer comprises cells that are wild type for EGFR. For example, the cancer may predominantly comprise cells that are wild type for EGFR. In certain embodiments, the cancer comprises cells that are mutant for EGFR. For example, the cancer may predominantly comprise cells that are mutant for EGFR. In certain embodiments, the cancer comprises cells that express high levels of EGFR. For example, the cancer may predominantly comprise cells that express high levels of EGFR. In certain embodiments, the cancer comprises cells that express low or moderate levels of EGFR. For example, the cancer may predominantly comprise cells that express low or moderate levels of EGFR. In certain embodiments, the cancer comprises cells that express high levels of MUC1. For example, the cancer may predominantly comprise cells that express high levels of MUC1. In certain embodiments, the cancer comprises cells that express low or moderate levels of MUC1. For example, the cancer may predominantly comprise cells that express low or moderate levels of MUC1.

In certain embodiments, the step of administering the immunoconjugate to the mammalian subject comprises administration by a systemic route, for example, an intravenous route or a subcutaneous route.

Depending upon the circumstances, tumor growth is reduced relative to a reference level after administration of the immunoconjugate to the mammalian subject. For example, tumor growth may regress partially or completely after the administration of the immunoconjugate to the mammalian subject.

In certain embodiments, the step of administering comprises administering at least two doses of the immunoconjugate, wherein the at least two doses collectively comprise a therapeutically effective amount. In certain embodiments, the step of administering comprises administering a single dose of the immunoconjugate that comprises a therapeutically effective amount.

In any one of the foregoing embodiments, the scFv of the first polypeptide may bind to a MUC1 epitope whose sequence comprises TRPAP (SEQ ID NO:27).

### BRIEF DESCRIPTION OF THE DRAWINGS

FIG. 1A shows the structure of SC239, a linker-drug molecule used in the synthesis of Molecule 1 (a bispecific anti-MUC1/EGFR antibody-drug conjugate of the present disclosure). SC239 comprises a DBCO group, a Val-Cit-PABA cleavable linker, and 3-aminophenyl-hemiasterlin. FIG. 1B shows a schematic depicting the structure of an exemplary antibody-drug conjugate of the present disclosure. In Molecule 1, n (the number of SC239 moieties) is 4.

FIG. 1C is a schematic depicting the structure of an exemplary MUC1/EGFR bispecific antibody in accordance with the present disclosure.

FIG. 2 shows in vitro cell killing curves of bispecific anti-MUC1/EGFR ADC (Molecule 1) and of control molecules on cells having various combinations of MUC1 and EGFR expression levels. Molecule 1 is H02/hC225-SC239, an ADC comprising a bispecific anti-MUC1/EGFR antibody conjugated to 3-aminophenyl-hemiasterlin. Molecules 2, 3, and 4 are monospecific ADCs having the same drug, drug-antibody-ratio (approximately 4), and linker used in Molecule 1. Molecule 2 is 1992-H02-SC239, an anti-MUC1 ADC. Molecule 3 is hC225-SC239, an anti-EGFR ADC. Molecule 4 is aGFP-SC239, an anti-GFP ADC. Molecule 9 is cetuximab, an anti-EGFR antibody. Tested cells were: (1) MDA-MB-468 breast cancer cells (MUC1+/EGFR+++), WISH cervical cancer cells (MUC1+++/EGFR+), OVCAR-3 ovarian cancer cells (MUC1++/EGFR+), HepG2 liver cancer cells (MUC1+/−/EGFR+/−), and CHO-k (Chinese Hamster Ovary cells; MUC1−/EGFR−). All graphs are presented as mean of triplicate values±SD.

FIG. 3 shows in vitro cell killing curves of the bispecific anti-MUC1/EGFR ADC Molecule 1 on Hekn cells (primary normal human epidermal keratinocyte, neonatal), MDA-MB-468 cancer cells, OVCAR-3 cancer cells and MCF-10A cells. All graphs are presented as mean of triplicate values±SD.

FIGS. 4A and 4B shows graphs of mean fluorescence intensity representative of internalization and trafficking to acidic compartments such as lysosomes of pHrodo™ labeled antibodies as assessed in two cancer cell lines. Internalization of pHrodo™ labeled (1) H02/hC225 SEED (Molecule 10), a bispecific anti-MUC1/EGFR antibody; (2) H02 IgG1 (Molecule 11), an anti-MUC1 antibody; (3) cetuximab (Molecule 9), an anti-EGFR antibody, and (4) rituximab (control Ab) were assessed in MDA-MB-468 (FIG. 4A) and OVCAR-3 (FIG. 4B) cells. Internalization into acidic cell compartments is represented by mean intensity of pHrodo signal vs. time points of measurement. All graphs are presented as mean of duplicate values±SD.

FIG. 5 shows in vitro cell killing curves of the bispecific anti-MUC1/EGFR ADC Molecule 1, together with monospecific control ADCs (Molecules 2 and 3; see description for FIG. 2) on different non-small cell lung cancer (NSCLC) cells. Shown is one representative experiment out of n=1-4 individual experiments. All graphs are presented as mean of triplicate values±SD.

FIG. 6 shows in vitro cell killing curves of bispecific anti-MUC1/EGFR ADC Molecule 1 and EGFR tyrosine kinase inhibitors (TKIs) on NSCLC cells NCI-H292 (left panel) and NCI-H1975 (right panel). Shown is one representative experiment out of n=3-4 individual experiments. All graphs are presented as mean of triplicate values±SD. Molecule 1 is H02/hC225-SC239, Molecule 12 is erlotinib, Molecule 13 is gefitinib, Molecule 14 is afatinib, and Molecule 15 is osimertinib.

FIG. 7 is a graph illustrating the plasma concentration-time profile of Molecule 1 following an IV bolus administration of a 5 mg/kg dose in CB17 SCID mice and Sprague-Dawley rats.

FIGS. 8A and 8B are graphs illustrating body weight change in mice bearing WISH tumor xenografts after being administered a single injection of Molecule 1 at different doses in two independent studies. (FIG. 8A: Study 1, with vehicle and 0.1 mg/kg, 0.3 mg/kg, 0.75 mg/kg, and 1.5 mg/kg doses; FIG. 8B: Study 2, with vehicle and 1.25 mg/kg, 2.5 mg/kg, and 5 mg/kg doses.)

FIGS. 9A and 9B are graphs illustrating tumor growth curves (FIG. 9A) and scatter plots (FIG. 9B) with final tumor sizes on day 21 in mice bearing WISH tumor xenografts after being administered a single injection of Molecule 1 at different doses (Study 1).

FIG. 10 is a graph illustrating tumor growth curves in mice bearing WISH tumor xenografts after being administered a single injection of Molecule 1 at different doses (Study 2).

FIG. 11 is a graph illustrating body weight change in mice bearing OVCAR-3 tumor xenografts after being administered a single injection of Molecule 1 at different doses.

FIGS. 12A and 12B are graphs illustrating tumor growth curves (FIG. 12A) and scatter plots (FIG. 12B) with final tumor sizes on day 28 in mice bearing OVCAR-3 tumor xenografts after being administered a single injection of Molecule 1 at different doses.

FIG. 13 is a graph illustrating body weight change in mice bearing MDA-MB-468 tumor xenografts after being administered a single injection of Molecule 1 at different doses.

FIG. 14 is a graph illustrating tumor growth curves in mice bearing MDA-MB-468 tumor xenografts after being administered a single injection of Molecule 1 at different doses.

FIGS. 15A and 15B are graphs illustrating tumor growth curves (FIG. 15A) in mice bearing the NSCLC patient derived xenografts LUX089 after being administered a single injection of Molecule 1 at different doses and the animal weight during the experiment (FIG. 15B).

FIG. 16A is a graph illustrating tumor growth curves in mice bearing NSCLC patient-derived xenografts after being administered the bispecific ADC Molecule 1 as compared to mice administered monospecific EGFR and MUC1 ADCs (Molecules 3 and 2, respectively, as described in the description for FIG. 2).

FIG. 16B is a graph illustrating the percent tumor volume change (TV %) induced by the bispecific ADC Molecule 1 and the monospecific EGFR and MUC1 ADCs in the NSCLC patient-derived xenograft models LUX019, LUX003 and LUX089 at the same dose.

FIGS. 17A and 17B are graphs illustrating tumor growth curves in mice bearing the NSCLC patient derived xenografts after being administered the same total dose of 8 mg/kg of Molecule 1 but using different treatment schedules.

FIGS. 18A, 18B, and 18C are graphs illustrating the percent tumor volume change (TV %) induced by a single 8 mg/kg dose of bispecific ADC Molecule 1 in a variety of patient-derived xenograft models from NSCLC, esophageal squamous cell carcinoma, and head and neck squamous cell carcinoma.

FIG. 19A depicts the structure of a MUC1 peptide in complex with H02-scFv. Dotted lines indicate hydrogen bonds between the MUC1 peptide and the H02-scFv.

FIG. 19B depicts details of the MUC1 peptide-H02-scFv interaction. Dotted lines indicate hydrogen bond between the MUC1 peptide (top part of complex) and the H02-scFv molecule (bottom part of complex).

FIG. 20 depicts a sequence alignment of heavy chain variable sequences from parent antibody HT186-D11 and from antibodies obtained during affinity maturation (see Example 1.) Amino acid residues corresponding to Chothia complementarity-determining regions (CDRs) are demarcated in black boxes. Amino acid residues corresponding to Kabat CDRs are highlighted in yellow.

### DETAILED DESCRIPTION

MUC1, a Type I transmembrane glycoprotein, is expressed on many cancer cells, but also exhibits some expression in normal cells. In tumor cells, MUC1 co-localizes and interacts with EGFR, and their interaction blocks ligand-activated EGFR degradation. The bispecific antibody-drug conjugates disclosed herein target both MUC1 and EGFR. By targeting both MUC1 and EGFR with the same antibody, the presently disclosed immunoconjugates not only enhance antibody internalization and tumor growth inhibition or reduction in tumor growth, they also enable higher specificity of binding to cancer cells, which may thereby reduce effects on normal cells.

The presently disclosed bispecific anti-MUC1/EGFR antibody-drug conjugates (ADCs) demonstrate therapeutic effects across a range of cancers, varying in tissue type, expression patterns for MUC1 and EGFR, and EGFR mutational status. Moreover, bispecific anti-MUC1/EGFR ADCs disclosed herein demonstrated superior tumor growth inhibition or reduction as compared to monospecific ADCs in various non-small cell lung cancer (NSCLC) patient-derived xenograft models.

### Definitions

As used herein, the terms “about,” “approximately,” and “comparable to,” when used herein in reference to a value, refer to a value that is similar to the referenced value in the context of that referenced value. In general, those skilled in the art, familiar with the context, will appreciate the relevant degree of variance encompassed by “about,” “approximately,” and “comparable to” in that context. For example, in some embodiments, the terms “about,” “approximately,” and “comparable to” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains.

An “antigen binding fragment” of an antibody, or “antibody fragment” comprises a portion of an intact antibody, which portion is still capable of antigen binding. Typically, such a portion comprises the variable region of the antibody. Papain digestion of antibodies produce two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (CHI). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and that is still capable of cross-linking antigen. Fab′ fragments differ from Fab fragments by having a few additional residues at the carboxy terminus of the C_{H}1 domain, including one or more cysteines from the antibody hinge region. Fab′-SH designates an Fab′ in which the cysteine residue(s) of the constant domains bear a free thiol group. F(ab′)₂ antibody fragments originally were produced as pairs of Fab′ fragments having hinge cysteines between them. Other chemical couplings of antibody fragments are also known.

An Fc fragment comprises the carboxy-terminal portions of both heavy chains held together by disulfides. The effector functions of antibodies are determined by sequences in the Fc region, the region which is also recognized by Fc receptors (FcR) found on certain types of cells.

As used herein, “polypeptide” refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

As used herein, the phrase “reference level” generally refers to a level considered “normal” for comparison purposes, e.g., a level of an appropriate control. For example, in the context of tumor growth inhibition or reduction, a “reference level” may refer to the level of tumor growth expected in a subject not receiving a therapeutic agent of interest (e.g., the level of tumor growth in a subject before the subject is administered a therapeutic agent of interest, or the level of tumor growth in another subject who is not receiving a therapeutic agent of interest), or in a subject receiving a treatment (e.g., the current standard of care) other than the therapeutic agent of interest. A reference level may be determined contemporaneously or may be predetermined, e.g., known or deduced from past observations.

As used herein, the phrases “therapeutically effective amount” and “effective amount” are used interchangeably and refer to an amount effective, at dosages and for periods of time necessary, to achieve a desired therapeutic result. A therapeutically effective amount may vary according to factors such as the type of disease (e.g., cancer), disease state, age, sex, and/or weight of the individual, and the ability of an immunoconjugate (or pharmaceutical composition thereof) to elicit a desired response in the individual. An effective amount may also be an amount for which any toxic or detrimental effects of the immunoconjugate or pharmaceutical composition thereof are outweighed by therapeutically beneficial effects.

As used herein, to “treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition (e.g., of a primary cancer and/or of a secondary metastases); delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### Immunoconjugates

### Bispecific Anti-MUC1/EGFR Antibodies

Immunoconjugates comprising bispecific anti-MUC1/EGFR antibodies of the present disclosure generally comprise (i) a first polypeptide comprising a first engineered Fc domain and a single-chain Fv (scFv), wherein the scFv binds to MUC1; (ii) a second polypeptide comprising a second engineered Fc domain and a heavy chain of an Fab fragment, and (iii) a third polypeptide comprising a light chain of the Fab fragment, wherein the second and third polypeptide chains together define an Fab fragment that binds EGFR.

As used herein, the term “Fc domain” refers to a C_{H}2 domain and a C_{H}3 domain of an immunoglobulin. Thus, a homodimer or heterodimer of two Fc domains is an Fc fragment. Fc domains used in accordance with the disclosure may be engineered in the sense that they (1) comprise an engineered C_{H}3 domain (as described herein) and/or (2) comprise one or more non-natural amino acids.

As used herein, the term “scFv” is used in accordance with its common usage in the art to refer to a single chain in which the V_{H} domain and the V_{L} domain from an antibody are joined, typically via a linker.

As used herein, the term “Fab fragment” is used in accordance with its common usage in the art. Fab fragments typically comprise an entire light chain (V_{L} and C_{L}1 domains), the variable region domain of the heavy chain (V_{H}), and the first constant domain of one heavy chain (C_{H}1).

Generally, the first and second polypeptides each comprise at least one non-natural amino acid at a predetermined site or sites intended to be used for conjugation. Non-natural amino acid may be located, e.g., in an Fc domain, in the heavy chain of an Fab domain, or both. Non-limiting examples of suitable non-natural amino acids include p-acetyl-L-phenylalanine, O-methyl-L-tyrosine, 3-methyl-phenylalanine, O-4-allyl-L-tyrosine, 4-propyl-L-tyrosine, fluorinated phenylalanine, isopropyl-L-phenylalanine, p-azido-L-phenylalanine, p-acyl-L-phenylalanine, p-benzoyl-L-phenylalanine, p-iodophenylalanine, p-bromophenylalanine, p-amino-L-phenylalanine, isopropyl-L-phenylalanine, p-propargyloxyphenylalanine, and p-azidomethyl-L-phenylalanine (see, e.g., U.S. Pat. No. 9,732,161). In some embodiments, the non-natural amino acid is para-azidomethyl-L-phenylalanine (pAMF).

In the first polypeptide, the engineered Fc domain may be fused to the scFv, e.g., with a hinge region intervening between the C_{H}2 domain of the engineered Fc domain and the V_{H} domain of the scFv. In some embodiments, the first polypeptide has an amino acid sequence at least 99% identical to that set forth in SEQ ID NO:1. For example, the first polypeptide may have an amino acid sequence that is 100% identical to that set forth in SEQ ID NO:11.

In the second polypeptide, the engineered Fc domain may be fused to the heavy chain of the Fab, e.g., with a hinge region intervening between the C_{H}2 domain of the engineered Fc domain and the C_{H}1 domain of the Fab fragment. In some embodiments, the second polypeptide has an amino acid sequence at least 99% identical to that set forth in SEQ ID NO:2. For example, the second polypeptide may have an amino acid sequence that is 100% identical to that set forth in SEQ ID NO:12.

In some embodiments, the third polypeptide has an amino acid sequence at least 99% identical to that set forth in SEQ ID NO:3. In some embodiments, the third polypeptide has an amino acid sequence that is 100% identical to that set forth in SEQ ID NO:3.

Typically, the first polypeptide and second polypeptide are covalently linked by one or more disulfide bonds formed between the first engineered Fc domain and the second engineered Fe domain. The second polypeptide and the third polypeptide are also typically covalently linked by one or more disulfide bonds formed between the heavy chain of the second polypeptide and the light chain of the third polypeptide.

In some embodiments, the scFv of the first polypeptide binds to a MUC1 epitope whose sequence comprises TRPAP (SEQ ID NO:27).

In some embodiments, the bispecific antibody is devised using a strand-exchange engineered domains (SEED)-based C_{H}3 heterodimer platform, as described, e.g., in U.S. Pat. Nos. 8,891,912 and 9,505,848. In this platform, each SEED-C_{H}3 domain comprises alternating segments of human IgA and IgG sequences. The “AG SEED” refers to a C_{H}3 domain that has an IgA1 sequence segment on the N-terminal end, while the “GA seed” refers to a C_{H}3 that has an IgG1 sequence segment on the N-terminal end. Each Fc heterodimer of a SEEDbody antibody comprises an AG SEED paired with a GA SEED.

Once constructs are designed for each chain (e.g., the first polypeptide, the second polypeptide, and the third polypeptide) of the bispecific anti-MUC1/EGFR antibody, constructs may also be mutagenized for the purpose of introducing non-natural amino acids (as discussed herein) at specific sites to be used as conjugation sites. These constructs may be expressed using any of a variety of expression systems known in the art.

In some embodiments, bispecific anti-MUC1/EGFR antibodies are produced using a cell-free system. Bispecific anti-MUC1/EGFR antibodies may have certain features reflecting how they were produced. For example, antibodies produced in a cell-free system may be aglycosylated and may lack effector functions.

Bispecific anti-MUC1/EGFR antibodies may optionally be purified before undergoing additional steps, such as conjugation.

### Hemiasterlin Moieties and Molecules

Hemiasterlin is a tri-peptide isolated from marine sponges that binds to the vinca binding site on tubulin. Hemiasterlin may thereby inhibit or reduce tubulin polymerization, which can trigger mitotic arrest and apoptosis. As used herein, term “hemiasterlin molecule” refers to a hemiasterlin or a hemiasterlin derivative that retains at least some function of hemiasterlin (e.g., tubulin-binding). The term “hemiasterlin moiety” refers to a hemiasterlin molecule that has been conjugated to another molecule. In some embodiments, the hemiasterlin derivative is 3-aminophenyl-hemiasterlin.

As described further herein, the number of hemiasterlin moieties per immunoconjugate may be controlled by using a site-specific conjugation method in which hemiasterlin moieties are conjugated to non-natural amino acids inserted at particular sites within a chain of the bispecific antibody (see, e.g., International Patent Publication WO 2019/055931.)

In some embodiments, each immunoconjugate has a plurality of hemiasterlin moieties, for example, 2, 3, 4, 5, 6, hemiasterlin moieties. In certain embodiments, the immunoconjugate contains four hemiasterlin moieties.

### Conjugation and Linkers

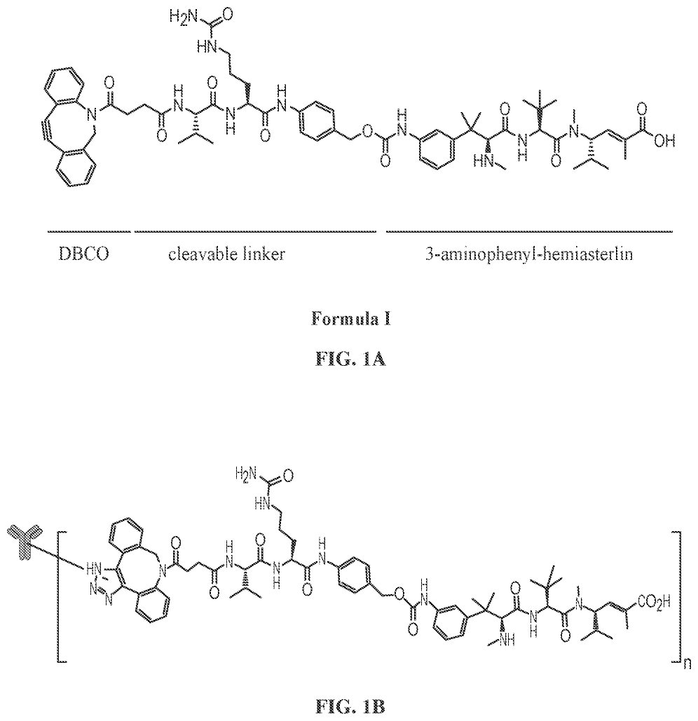

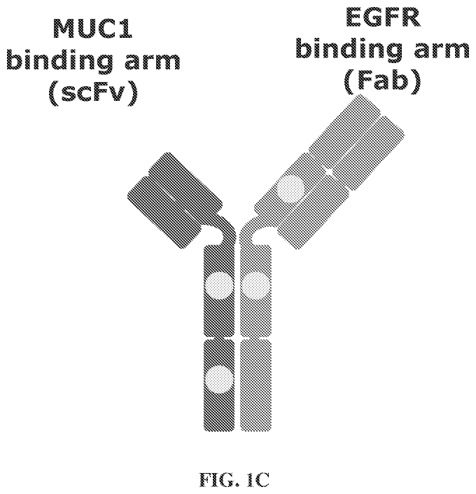

Conjugation reactions may be performed using functionalized linker-drug molecule, wherein the linker is a cleavable linker. Copper-free click chemistry reactions may be used with certain functionalized groups. In some embodiments, immunoconjugates are generated by reacting bispecific anti-MUC1/EGFR antibodies with the SC239 linker-drug molecule whose structure is depicted in FIG. 1A. SC239 comprises a 3-aminophenyl-hemiasterlin and a cleavable valine citrulline p-aminobenzylalcohol (Val-Cit-PABA) linker functionalized with dibenzocyclooctyne (DBCO) (see, e.g., WO 2019/0055931 A1.)

### Conjugation Sites

Generally, non-natural amino acid residues are introduced into the first, second, or third polypeptide at sites that may be used to conjugate one or more moieties, e.g., hemiasterlin moieties. Thus, the locations of non-natural amino acid residues may correspond to conjugation sites.

In some embodiments, the first engineered Fc domain comprises two non-natural amino acid residues, for example, at heavy chain positions F241 and F404 according to the EU index. In some embodiments, the first engineered Fc domain comprises no more than two non-natural amino acid residues.

In some embodiments, the single-chain scFv on the first polypeptide comprises a non-natural amino acid residue, for example, within the heavy chain variable domain at position S7, T22, or a combination thereof according to the EU index.

In some embodiments, the second engineered Fc domain comprises a non-natural amino acid residue, for example, at heavy chain position F241 according to the EU index. In some embodiments, the second engineered Fc domain comprises no more than one non-natural amino acid residue.

In some embodiments, the Fab fragment comprises a non-natural amino acid residue. In some embodiments, the heavy chain of the Fab fragment within the second polypeptide comprises a non-natural residue, for example, at heavy chain position S136, Y180, S190, or a combination thereof according to the EU index. In some embodiments, the Fab fragment comprises no more than one non-natural amino acid residue. In some embodiments, the heavy chain of the Fab fragment within the second polypeptide comprises a non-natural amino acid residue at heavy chain position Y180 according to the EU index.

### Exemplary Conjugates

In certain embodiments, immunoconjugates have the structure shown in Formula II:

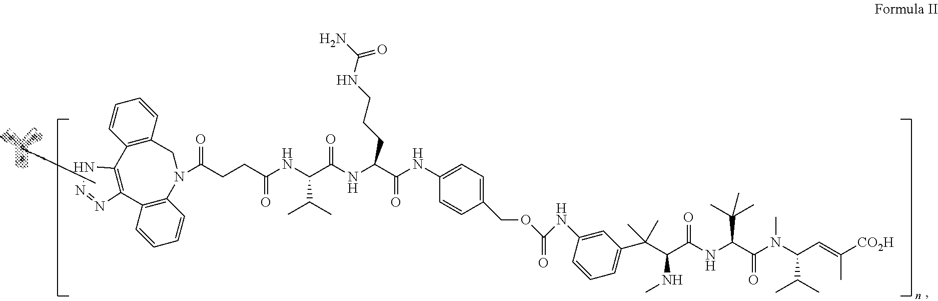

wherein n is greater than 1. In some embodiments, n is at least 2, at least 3, at least 4, at least 5, at least 6, at least 7, at least 8, at least 9, at least 10, or more. In some embodiments, n is 2, 3, 4, 5, 6, 7, 8, 9, or 10. In some embodiments, n is 4.

### Pharmaceutical Compositions

In certain embodiments, provided immunoconjugates are incorporated together with one or more pharmaceutically acceptable carriers into a pharmaceutical composition suitable for administration to a subject. As used herein, “pharmaceutically acceptable carrier” refers to any of a variety of solvents, dispersion media, coatings, antibacterial and antifungal agents, isotonic and absorption delaying agents, and the like that are physiologically compatible. Examples of pharmaceutically acceptable carriers include, but are not limited to, water, saline, phosphate buffered saline, dextrose, glycerol, ethanol and the like, as well as combinations thereof.

In some embodiments, pharmaceutical compositions comprise one or more tonicity agents or stabilizers. Non-limiting examples of such tonicity agents or stabilizers include sugars (e.g., sucrose), polyalcohols (e.g., mannitol or sorbitol), and sodium chloride.

In some embodiments, pharmaceutical compositions comprise one or more bulking agents and/or lyoprotectants (e.g., mannitol or glycine), buffers (e.g., phosphate, acetate, or histidine buffers), surfactants (e.g., polysorbates), antioxidants (e.g., methionine), and/or metal ions or chelating agents (e.g., ethylenediaminetetraacetic acid (EDTA)).

In some embodiments, pharmaceutical compositions comprise one or more auxiliary substances such as wetting or emulsifying agents, preservatives (e.g., benzyl alcohol) or buffers, which may enhance the shelf life and/or effectiveness of immunoconjugates disclosed herein.

Pharmaceutical compositions may be provided in any of a variety of forms. These include, for example, liquid, semi-solid and solid dosage forms, such as liquid solutions (e.g., injectable and infusible solutions), dispersions or suspensions, tablets, pills, powders, liposomes and suppositories. Suitability of certain forms may depend on the intended mode of administration and therapeutic application.

In some embodiments, pharmaceutical compositions are in the form of injectable or infusible solutions.

Pharmaceutical compositions are typically sterile and stable under conditions of manufacture, transport, and storage. Pharmaceutical compositions may be formulated as, for example, a solution, microemulsion, dispersion, liposome, or other ordered structure. In some embodiments, a pharmaceutical composition is formulated as a structure particularly suitable for high drug concentration. For example, sterile injectable solutions can be prepared by incorporating a therapeutic agent (e.g., immunoconjugate) in a desired amount in an appropriate solvent with one or a combination of ingredients enumerated herein, optionally followed by sterilization (e.g., filter sterilization). Generally, dispersions may be prepared by incorporating an immunoconjugate into a sterile vehicle that contains a basic dispersion medium and other ingredient(s) such as those additional ingredients mentioned herein. In the case of sterile powders for the preparation of sterile injectable solutions, examples of preparation methods include vacuum drying and freeze-drying to yield a powder of the immunoconjugate and any additional desired ingredient(s), e.g., from a previously sterile-filtered solution thereof.

Proper fluidity of a solution can be maintained, for example, by the use of a coating such as lecithin, by maintaining certain particle sizes (e.g., in the case of dispersions), and/or by using surfactants. Prolonged absorption of injectable compositions can be brought about, e.g., by including in the composition an agent that delays absorption (for example, monostearate salts and/or gelatin).

### Methods of Treatment

Methods of treating cancer disclosed herein generally comprise a step of administering a therapeutically effective amount of an immunoconjugate (or pharmaceutical composition thereof) of the present disclosure to a mammalian subject (e.g., a human subject) in need thereof. In some embodiments, the subject is diagnosed as having cancer.

Therapeutically effective amounts may be administered via a single dose or via multiple doses (e.g., at least two, at least three, at least four, at least five, at least six, at least seven, at least eight, at least nine, or at least ten doses). When administered via multiple doses, any of a variety of suitable therapeutic regimens may be used, including administration at regular intervals (e.g., once every other day, once every three days, once every four days, once every five days, thrice weekly, twice weekly, once a week, once every two weeks, once every three weeks, etc.).

The dosage regimen (e.g., amounts of each therapeutic, relative timing of therapies, etc.) that is effective in methods of treatment may depend on the severity of the disease or condition and the weight and general state of the subject. For example, the therapeutically effective amount of a particular composition comprising a therapeutic agent applied to mammals (e.g., humans) can be determined by the ordinarily-skilled artisan with consideration of individual differences in age, weight, and the condition of the mammal. Therapeutically effective and/or optimal amounts can also be determined empirically by those of skill in the art. In some embodiments, subjects are administered a dose between 0.4 mg/kg every 3 days to 20 mg/kg every 3 days. Immunoconjugates and pharmaceutical compositions thereof may be administered by any of a variety of suitable routes, including, but not limited to, systemic routes such as parenteral (e.g., intravenous or subcutaneous) or enteral routes.

In certain embodiments, the subject is diagnosed with cancer.

### Cancers

In some embodiments, the cancer comprises a solid tumor. For example, the cancer may be selected from the group consisting of breast cancer, lung cancer, esophageal cancer, head and neck cancer, cervical cancer, ovarian cancer, and gastric cancer. In some embodiments, the cancer is breast cancer, for example, triple negative breast cancer. In some embodiments, the cancer is lung cancer, for example, a non-small cell lung cancer (NSCLC), such as an NSCLC comprising an adenocarcinoma and/or a squamous cell carcinoma. In some embodiments, the cancer is esophageal cancer, for example, squamous esophageal cancer. In some embodiments, the cancer is head and neck cancer, for example, head and neck squamous cell carcinoma. In some embodiments, the cancer is cervical cancer. In some embodiments, the cancer is ovarian cancer. In some embodiments, the cancer is gastric cancer. In some embodiments, the cancer is mesothelioma. In some embodiments, the solid tumor is metastatic.

In some embodiments, the cancer comprises a non-solid tumor, for example, multiple myeloma.

In some embodiments, the cancer comprises cells that are genotypically wild type for EGFR.

In some embodiments, the cancer comprises cells that express a mutant form of EGFR. Examples of EGFR mutations associated with cancers include, but are not limited to, deletion mutations (e.g., exon 19 deletions), point mutations (e.g., L858R mutations), insertion mutations (e.g., exon 20 insertions), and gene amplifications. Some EGFR mutations cause altered EGFR expression levels, e.g., overexpression of EGFR. Some EGFR mutations are associated with poor prognosis and/or resistance to targeted EGFR inhibitors.

In some embodiments, the cancer comprises cells that are genotypically wild type for MUC1.

In some embodiments, the cancer comprises cells that express a mutant form of MUC1. Examples of MUC1 mutations associated with cancers include, but are not limited to, point mutations (e.g., T112P). Some MUC1 mutations cause altered MUC1 expression levels, e.g., overexpression of MUC1, which has been associated with poor prognosis for some cancers.

Cancer cells may be characterized as having low/moderate or high levels of EGFR expression, as well as low/moderate or high levels of MUC1 expression (e.g., low/moderate levels of EGFR and low/moderate levels of MUC1; high levels of EGFR and low/moderate levels of MUC1; low/moderate levels of EGFR and high levels of MUC1; and high levels of EGFR and high levels of MUC1). Numerical levels that correspond to low, moderate, or high levels (including overexpression) of a gene product may vary depending on the particular gene product and may be assessed by any of a variety of means, such as assessment of surface expression (e.g., cell surface staining by FACS), protein expression by IHC, transcript levels (e.g., by RNASeq or qPCR), etc.

In some embodiments, a cancer cell that expresses “high levels of MUC1” is a cancer cell that expresses MUC1 at levels characterized by one or more of (1) median fluorescence intensity (MFI) ratio (MFI anti-MUC1 antibody/MFI isotype) of more than 200 (e.g., as determined by FACS, e.g., as described in Example 7); (2) comparable to or higher than that expressed by WISH (cervical cancer) cells grown in standard cell culture conditions for WISH cells; and (3) higher than that expressed by OVCAR-3 (ovarian cancer) cells grown in standard cell culture conditions for OVCAR-3 cells.

In some embodiments, a cancer cell that expresses “moderate levels of MUC1” is a cancer cell that expresses MUC1 at levels characterized by one or more of (1) median fluorescence intensity (MFI) ratio (MFI anti-MUC1 antibody/MFI isotype) of more than 100 but no more than 200 (e.g., as determined by FACS, e.g., as described in Example 7); (2) comparable to that expressed by OVCAR-3 cells grown in standard cell culture conditions for OVCAR-3 cells; and (3) (i) higher than that expressed by MDA-MD-468 (breast cancer) cells grown in standard cell culture conditions for MDA-MD-468 cells and (ii) lower than that expressed by WISH cells grown in standard cell culture conditions for WISH cells.

In some embodiments, a cancer cell that expresses “low levels of MUC1” is a cancer cell that expresses MUC1 at levels characterized by one or more of (1) median fluorescence intensity (MFI) ratio (MFI anti-MUC1 antibody/MFI isotype) of up to 100 (e.g., as determined by FACS, e.g., as described in Example 7); (2) comparable to or lower than that expressed by MDA-MD-468 cells grown in standard cell culture conditions for MDA-MD-468 cells; (3) lower than that expressed by OVCAR-3 cells grown in standard cell culture conditions for OVCAR-3 cells; (4) comparable to or lower than that expressed by NCI-H292 (non-small cell lung cancer) cells; (5) comparable to or lower than that expressed by HCC827 (non-small cell lung cancer) cells; and (6) comparable to or lower than that expressed by NCI-H1975 (non-small cell lung cancer) cells.

In some embodiments, a cancer cell that expresses “high levels of EGFR” is a cancer cell that expresses EGFR at levels characterized by one or more of (1) median fluorescence intensity (MFI) ratio (MFI anti-EGFR antibody/MFI isotype) of more than 200 (e.g., as determined by FACS, e.g., as described in Example 7); (2) comparable to or higher than that expressed by MDA-MD-468 cells grown in standard cell culture conditions for MDA-MD-468 cells; (3) comparable to or higher than that expressed by HCC827 (non-small cell lung cancer) cells grown in standard cell culture conditions for HCC827 cells; and (4) higher than that expressed by NCI-H292 (non-small cell lung cancer) cells grown at standard cell culture conditions for NCI-H292 cells.

In some embodiments, a cancer cell that expresses “moderate levels of EGFR” is a cancer cell that expresses EGFR at levels characterized by one or more of (1) median fluorescence intensity (MFI) ratio (MFI anti-EGFR antibody/MFI isotype) of more than 100 but no more than 200 (e.g., as determined by FACS, e.g., as described in Example 7); (2) comparable to that expressed by NCI-H292 cells grown at standard cell culture conditions for NCI-H292 cells; (3) (i) higher than that expressed by WISH cells grown in standard cell culture conditions for WISH cells and (ii) lower than that expressed by MDA-MD-468 cells grown in standard cell culture conditions for MDA-MD-468 cells; (4) (i) higher than that expressed by OVCAR-3 cells grown in standard cell culture conditions for OVCAR-3 cells and (ii) lower than that expressed by MDA-MD-468 cells grown in standard cell culture conditions for MDA-MD-468 cells; and (5) (i) higher than that expressed by NCI-H1975 (non-small cell lung cancer) cells grown in standard cell culture conditions for NCI-H1975 cells and (ii) lower than that expressed by HCC827 cells grown in standard cell culture conditions for HCC827 cells.

In some embodiments, a cancer cell that expresses “low levels of EGFR” is a cancer cell that expresses EGFR at levels characterized by one or more of (1) median fluorescence intensity (MFI) ratio (MFI anti-EGFR antibody/MFI isotype) of up to 100 (e.g., as determined by FACS, e.g., as described in Example 7); (2) comparable to or lower than that expressed by WISH cells grown in standard cell culture conditions for WISH cells; (3) comparable to or lower than that expressed by OVCAR-3 cells grown in standard cell culture conditions for OVCAR-3 cells; (4) comparable to or lower than that expressed by NCI-H1975 cells grown in standard cell culture conditions for NCI-H1975 cells; and (5) lower than that of NCI-H292 cells grown in standard cell culture conditions for NCI-H292 cells.

In some embodiments, the cancer is heterogeneous with respect to one or more of EGFR mutant status, EGFR expression level, and MUC1 expression level. In some such heterogenous cancers, the cancer may predominantly comprise one or another cell type (with respect to EGFR mutant status, EGFR expression level, and/or MUC1 expression level). As used herein, a cancer is described as “predominantly” comprising a cell type when at least 55%, at least 60%, at least 65%, at least 70%, at least 75%, at least 80%, at least 85%, at least 90%, or at least 95% of the cancer's cells are of that cell type.

In some embodiments, administration results in a measurable improvement in the subject. For example, this improvement may include any or any combination of tumor growth inhibition (TGI), tumor growth reduction, tumor regression, inhibition or reduction of metastases, improved survival, or improvement in any clinical sign indicative of cancer status or progression. Tumor growth may be assessed by measures such as, e.g., estimated or measured tumor volumes. In some embodiments, tumor growth inhibition or reduction is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% (e.g., based on lower tumor volume relative to a reference, such as a reference value representative of a tumor volume in a subject receiving no treatment). In some embodiments, administration results in regression of the tumor, i.e. a decrease in size of a tumor or in extent of cancer in the body relative to the size at the commencement of a therapeutic regimen involving an immunoconjugate. This tumor regression may be partial (i.e., some of the tumor or cancer remains) or complete (e.g., the tumor volume reaches approximately zero and/or the tumor is no longer measurable or detectable).

### EXAMPLES

The following specific examples are to be construed as merely illustrative, and not limiting of the remainder of the disclosure in any way whatsoever.

### Example 1. Sequence Optimization and Affinity Maturation to Develop an Anti-MUC1 scFv Sequence

An anti-MUC1 scFv (H02) was developed by affinity maturation of anti-MUC1 antibody HT186-D11 (see Thie H. et al. PLoS One 201, 6, 1, e15921) using ribosome display selection. For the scFv library, CDRs H1, H2, H3 and L3 (SEQ ID NOs: 4, 5, 6, and 9) were targeted. scFv ribosome display selections were then performed against a biotinylated synthetic VNTR peptide of MUC (APDTRPAPGSTAPPAC-biotin) (SEQ ID NO:10).

Antibody variants were screened and characterized based, among other things, on binding to MUC1-expressing cells (WISH, MDA-MB-468, and OVCAR-3 cancer cells, with HepG2 cells as MUC1-negative controls) (see Example 4 for additional details), binding to a biotinylated synthetic VNTR peptide of MUC1 (APDTRPAPGSTAPPAC-biotin; SEQ ID NO:10), association kinetics, stability in storage, and cell killing of MUC-1 positive cells by a drug conjugate of the antibody variant (ADC).

ADCs were generated by site-specific conjugation using a cell-free expression system and conjugation to SC239 (a cleavable linker-hemiasterlin derivative) (see Example 3 for details regarding SC239.)

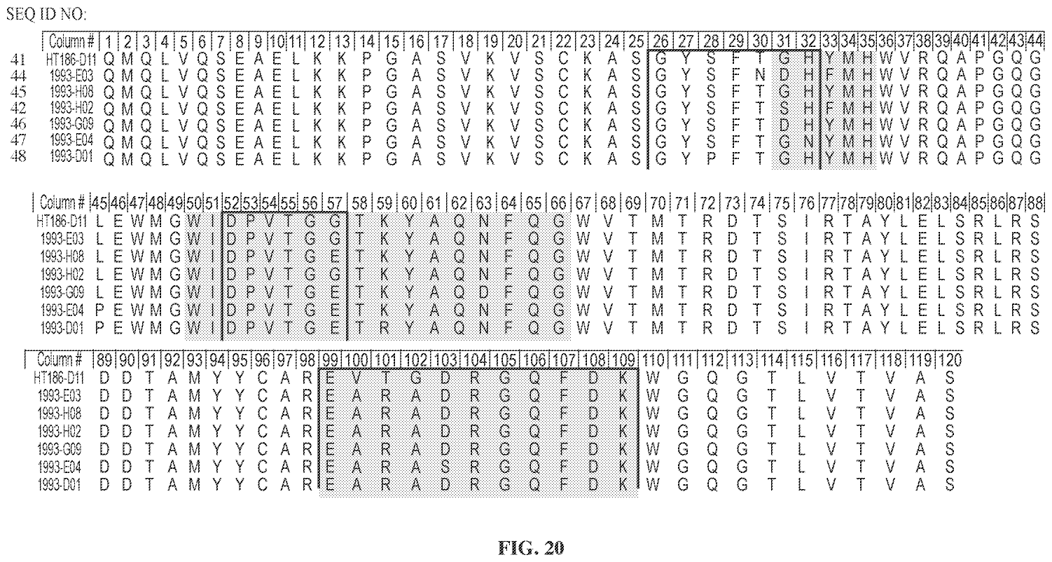

FIG. 20 depicts a sequence alignment of heavy chain variable sequences from parent antibody HT186-D11 and from antibodies obtained during affinity maturation. Amino acid residues corresponding to Chothia complementarity-determining regions (CDRs) are demarcated in black boxes. Amino acid residues corresponding to Kabat CDRs are shaded in gray.

Based on these studies, antibody variant “1993-H02” (hereinafter H02) was chosen and developed as an scFv. A summary of the H02 sequence's binding characteristics is provided in Table 1A; a summary of results from cell killing assays is provided in Table 1B.

TABLE 1A
H02 characterization for 1993-H02 with an HT186-D11 light chain

| | |
|---|---|
| Biacore, human MUC1-VNTR peptide | WISH, cell binding |

| KD (M): | kd (½) | Bmax | Kd (nM) |
|---|---|---|---|
| 9.01E−10 | 2.14E−04 | 128920 | 23.9 |

| Turbidity | Thermostability with | | Biacore, human MUC1-VNTR | |
|---|---|---|---|---|
| A600 | Tm1 (° C.) | Tm2 (° C.) | ka1 (1/Ms) | kd1 (1/s) |
| 0.15 | 62 | ND | 1.26E+05 | 1.70E−03 |

TABLE 1B
ADC cell killing assay results for 1993-H02 conjugated to SC239 (DAR = 4)

| WISH | | MDA-MB-468 | | OVCAR-3 | | HepG2 | |
|---|---|---|---|---|---|---|---|
| IC₅₀ (nM) | Span (%) | IC₅₀ (nM) | Span (%) | IC₅₀ (nM) | Span (%) | IC₅₀ (nM) | Span (%) |
| 1.1 | 94 | 1.7 | 92 | 1.9 | 93 | NK | NK |

NK = no killing

### Example 2. Construction of Bispecific Anti-MUC1/EGFR Antibodies

Bispecific anti-MUC1/EGFR antibodies were developed using a strand-exchange engineered domains (SEED)-based C_{H}3 heterodimer platform (see, e.g., as described in U.S. Pat. Nos. 8,891,912 and 9,505,848).

A bispecific antibody (hereinafter “Molecule 10”) was designed as a heterodimer of:
an anti-MUC1 scFv (H02) fused to a human IgG1 Fe (AG SEED); and
an anti-EGFR Fab (derived from humanized cetuximab) fused to a human IgG1 Fe (GA SEED).

Expression constructs encoding the anti-MUC1 scFvFc (AG SEED), the heavy chain of the anti-EGFR Fab fused to the IgG1 Fc (GA SEED), and the light chain of the anti-EGFR Fab were constructed. Upon protein expression and heterodimer formation, the resulting product is a bispecific anti-MUC1/EGFR antibody (H02/hC225 SEED, or “Molecule 10”).

For purposes of conjugation site optimization studies described in Example 3, similar methods were used to construct a similar bispecific anti-MUC1/EGFR antibody (D11/hC225). In D11/hC225, the anti-MUC1 arm was based on the HT186-D11 scFv (the parental sequence from which H02 was developed; see Example 1) fused to a human IgG1 Fc (AG SEED), and the anti-EGFR arm was based on the Fab of humanized cetuximab (hC225) fused to an IgG1 Fc (GA seed).

### Example 3. Synthesis of a Molecule 1, a Bispecific Anti-MUC1/EGFR Antibody Conjugated to 3-Aminophenyl-Hemiasterlin (“Bispecific Anti-MUC1/EGFR ADC”)

The XpressCF+™ (Sutro Biopharma) cell-free expression system and site-specific conjugation method (see, e.g., U.S. Pat. No. 9,732,161 and International Patent Publication No. WO 2019/055931 A1) was used to generate antibody-drug conjugates based on the bispecific anti-MUC1/EGFR antibody H02/hC225 SEED (Molecule 10) described in Example 1.

For initial experiments to optimize conjugation sites, the anti-MUC1 arm of D11/hC225 (AG SEED) and the heavy chain of the anti-EGFR arm of D11/hC225 (GA SEED) (see Example 1) were mutagenized by incorporating the non-natural amino acid para-azido methyl L-phenylalanine (pAMF) at TAG sites (amber stop codons). A series of mutants were generated for each arm (anti-MUC1 scFvFc (AG SEED) arm or anti-EGFR Fab(heavy chain)Fc (GA SEED) arm), each mutant having only one pAMF residue incorporated. The pAMF residues in each mutant arm were conjugated to a hemiasterlin derivative by copper-free click chemistry using SC239, which comprises a tubulin-targeting 3-aminophenyl hemiasterlin and a cleavable valine citrulline p-aminobenzylalcohol (Val-Cit-PABA) linker functionalized with dibenzocyclooctyne (DBCO) (see, e.g., WO 2019/0055931 A1.)

SC239 has the structure shown in Formula I:

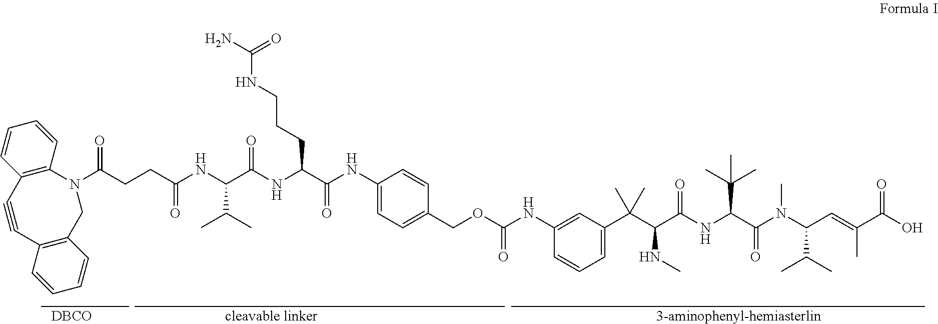

Conjugated anti-MUC1 scFvFc (AG) and anti-EGFR Fab(heavy chain)Fc (GA) arms were separately tested in vitro for binding to MUC1 and EGFR, respectively, and for MDA-MB-468 (human breast cancer) cell killing. Combinations of anti-MUC1 scFvFc (AG) and anti-EGFR Fab(heavy chain)Fc (GA) arms were also tested in vitro for binding to EGFR, binding to MUC1, and MDA-MB-468 cell killing. Factors affecting manufacturability, such as protein expression, yield, and thermostability, were also taken into consideration.

Based on results from conjugation site optimization studies, the following conjugation sites were chosen, with all positions numbered according to the EU index:
Heavy chain position F404 on the anti-MUC1 scFv;
Heavy chain position F241 on the anti-MUC1 scFv;
Heavy chain position F241 on the anti-EGFR Fab; and
Heavy chain position Y180 on the anti-EGFR Fab.

Using the these conjugation sites, an ADC having the structure of Formula II

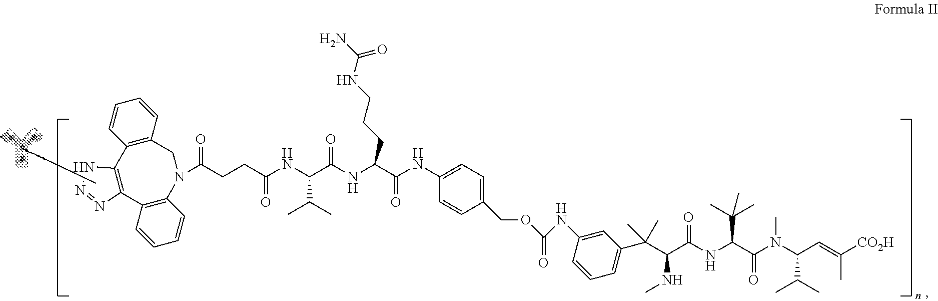

(also shown in FIG. 1B) (with n=4) was synthesized using sequences for the H02/hC225 SEED bispecific antibody (Molecule 10) described in Example 2. This ADC (hereinafter “Molecule 1”) has a drug-antibody-ratio of approximately 4 and comprises a bispecific antibody having an anti-MUC1 scFvFc (AG SEED), an anti-EGFR Fab(heavy chain)Fc (GA SEED), and an anti-EGFR Fab (light chain), the H02/hC225 SEED bispecific antibody being conjugated at each of the above-mentioned conjugation sites to a 3-aminophenyl-hemiasterlin molecule via the Val-Cit-PABA cleavable linker.

### Molecules Used Examples 4-20

Table 2 summarizes molecules used in the experiments described Examples 4-20.

In particular, Molecules 1, 2, and 3 are antibody-drug conjugates, generated as described in Example 2. Molecule 10 is a bispecific antibody generated as described in Example 2. Molecules 9 and 11 are mono-specific antibodies. Molecules 12-15 are small molecule EGFR tyrosine kinase inhibitors (TKIs) known in the art (see, e.g., Hirano et al., In vitro modeling to determine mutation specificity of EGFR tyrosine kinase inhibitors against clinically relevant EGFR mutants in non-small-cell lung cancer. Oncotarget 2015, 6, 38789-38803).

TABLE 2
Molecules used in Examples 4-16
Antibody-drug conjugates

| | | Drug- | |
|---|---|---|---|
| | Short name/ | antibody- | Full description and/or other |
| Molecule # | description | ratio | names |
| 1 | Anti- | DAR: 4 | Bispecific anti-MUC1/EGFR |
| | MUC1/ | | antibody conjugated to 3- |
| | EGFR | | aminophenyl-hemiasterlin via a |
| | ADC | | DBCO Val Cit PABA linker |
| | | | (H02/hC225-SC239) |
| 2 | Anti-MUC1 | DAR: 4 | Anti-MUC1 antibody |
| | ADC | | conjugated to 3-aminophenyl- |
| | | | hemiasterlin via a DBCO Val |
| | | | Cit PABA linker |
| | | | (1993-H02-SC239) |
| 3 | Anti- | DAR: 4 | Anti-EGFR antibody conjugated |
| | EGFR | | to 3-aminophenyl-hemiasterlin |
| | ADC | | via a DBCO Val Cit PABA |
| | | | linker |
| | | | (hC225-SC239) |
| 4 | Anti-GFP | DAR: 4 | Anti-GFP conjugated to 3- |
| | ADC | | aminophenyl-hemiasterlin via a |
| | | | DBCO Val Cit PABA linker |
| | | | (aGFP-SC239) |

Antibodies

| | | Full description and/or |
|---|---|---|
| Molecule # | Short name/description | other names |
| 9 | cetuximab (anti-EGFR antibody) | — |
| 10 | Bispecific anti-MUC1/EGFR | H02/hC225 |
| | antibody | |
| 11 | Anti-MUC1 antibody | H02 IgG1 |

Small molecule EGFR tyrosine kinase inhibitors (TKIs)

| Molecule # | Inhibitor |
|---|---|
| 12 | erlotinib |
| 13 | gefitinib |
| 14 | afatinib |
| 15 | osimertinib |

* According to EU index numbering system.

### Example 4. Bispecific Anti-MUC1/EGFR ADC Effectively Kills Cancer Cells In Vitro

To assess the effect of bispecific anti-MUC1/EGFR ADC on cancer cells, a cell killing assay was performed on Molecule 1 using various human cancer cells expressing varying levels of MUC1 and EGFR: MDA-MD-468 (breast cancer; MUC1+/EGFR+++), WISH (cervical cancer; MUC1+++/EGFR+), OVCAR-3 (ovarian cancer; MUC1++/EGFR+), and HepG2 (liver cancer; having low but non-zero expression of MUC1 and of EGFR) cells. Bispecific anti-MUC1/EGFR ADCs were also tested on non-cancerous CHO-k (Chinese Hamster Ovary; MUC1−/EGFR−) cells.

### Methods

WISH, OVCAR-3, HepG2, MDA-MB-468, and CHO-k cells were purchased from ATCC (American Type Culture Collection), and the cells were maintained in DMEM/F12 (1:1), high glucose (Corning®) supplemented with 10% heat-inactivated fetal bovine serum (Thermo Fisher Scientific®), 2 mM glutamax (Thermo Fisher Scientific®), and 1× Penicillin/streptomycin (Corning®).

Cytotoxicity effects of the ADC on cancer cells were measured with a cell proliferation assay. A total of 625 cells in a volume of 25 μL were seeded in a 384-well flat bottom white polystyrene plate the day before the actual assay started. ADC and free drugs were formulated at 2× starting concentration in cell culture medium and filtered through SpinX 0.22 μm cellulose acetate filtered 2 ml centrifuge tubes (Corning® Costar®). Filter sterilized samples were serial diluted (1:3) under sterile conditions and 25 μL of each dilution was added onto cells in triplicates. Plates were cultured at 37° C. in a CO₂ incubator for 120 hours. For cell viability measurement, 30 μL of Cell Titer-Glo® reagent (Promega™ Corp, Madison, WI) was added into each well, and plates processed as per product instructions. Relative luminescence was measured on an ENVISION® plate reader (Perkin-Elmer; Waltham, MA). Relative luminescence readings were converted to % viability using untreated cells as controls. Data was fitted with non-linear regression analysis, using log (inhibitor) versus response, variable slope, 4-parameter fit equation using GraphPad Prism. Data was expressed as % relative cell viability vs. dose of ADC in nanomolar with error bars indicating the Standard Deviation (SD) of the triplicates.

### Results

The cell killing activity of the bispecific anti-MUC1/EGFR ADC generated as described in Example 2 (Molecule 1) was evaluated on cells with varied expression levels of EGFR and MUC1 antigens. The following monospecific ADCs comprising the same drug (3-aminophenyl-hemiasterlin) were used as controls, along with an anti-EGFR antibody:
anti-MUC1 ADC (Molecule 2)
anti-EGFR ADC (Molecule 3)
commercial grade cetuximab (anti-EGFR antibody) (Molecule 9)
anti-GFP ADC (Molecule 4).

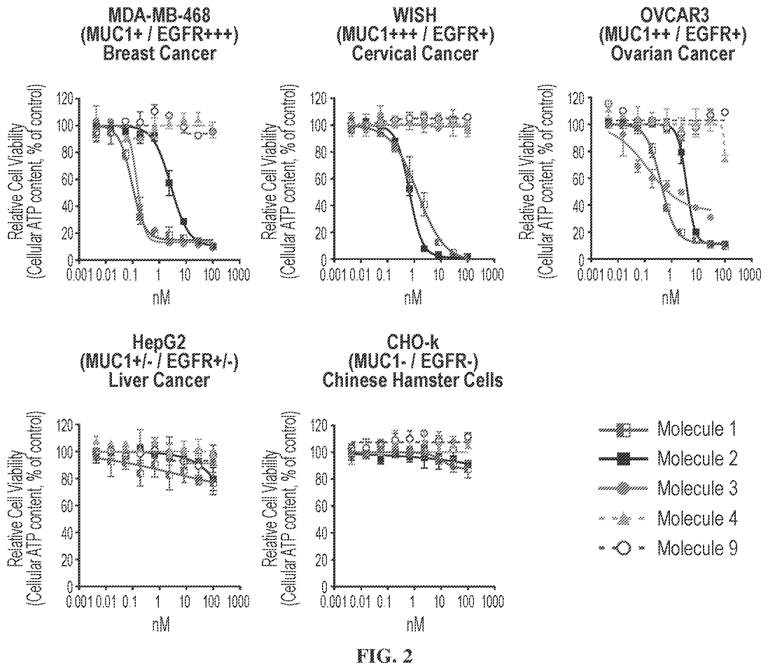

The cell killing curves (FIG. 2) and results are reported as IC₅₀ (the midpoint of the curve, or concentration at which 50% of the maximum inhibition was observed) as well as killing span (the total percentage of cells that are no longer viable relative to an untreated control at the maximum effect level of the test article, % efficacy) in (Table 3).

TABLE 3
IC₅₀ values and Killing Spans of ADCs and control molecules

| | | | MDA-MB-468 | | WISH | |
|---|---|---|---|---|---|---|
| Molecule No. | Sample | DAR | IC₅₀ (nM) | Span (%) | IC₅₀ (nM) | Span (%) |
| Molecule 1 | anti-MUC1/EGFR ADC | 3.7 | 0.09 | 85 | 1.4 | 98 |
| Molecule 2 | anti-MUC1 ADC | 3.84 | 2.7 | 90 | 0.65 | 99 |
| Molecule 3 | anti-EGFR ADC | 3.83 | 0.15 | 87 | NK | NK |
| Molecule 4 | anti-GFP ADC | 3.58 | NK | NK | NK | NK |
| Molecule 9 | cetuximab (anti- | NA | NK | NK | NK | NK |
| | EGFR antibody) | | | | | |

OVCAR-3

| Molecule No. | Sample | DAR | IC₅₀ (nM) | Span (%) |
|---|---|---|---|---|
| Molecule 1 | anti-MUC1/EGFR ADC | 3.7 | 0.41 | 88 |
| Molecule 2 | anti-MUC1 ADC | 3.84 | 3.6 | 89 |
| Molecule 3 | anti-EGFR ADC | 3.83 | 0.13 | 65 |
| Molecule 4 | anti-GFP ADC | 3.58 | NC | NC |
| Molecule 9 | cetuximab (anti- | NA | NK | NK |
| | EGFR antibody) | | | |

| | | | HepG2 | | CHO-K | |
|---|---|---|---|---|---|---|
| Molecule No. | Sample | DAR | IC₅₀ (nM) | Span (%) | IC₅₀ (nM) | Span (%) |
| Molecule 1 | anti-MUC1/EGFR ADC | 3.7 | NK | NK | NK | NK |
| Molecule 2 | anti-MUC1 ADC | 3.84 | NK | NK | NK | NK |
| Molecule 3 | anti-EGFR ADC | 3.83 | NK | NK | NK | NK |
| Molecule 4 | anti-GFP ADC | 3.58 | NK | NK | NK | NK |
| Molecule 9 | cetuximab (anti- | NA | NK | NK | NK | NK |
| | EGFR antibody) | | | | | |

DAR = drug-antibody ratio
* Estimated
NC = Not calculable due to incomplete dilution curve
NK = No Killing

In all three cancer cell lines co-expressing MUC1 and EGFR, Molecule 1 potently inhibited cell viability at high efficacy, independent of the MUC1 and EGFR expression levels.

On MDA-MB-468 cells, the anti-EGFR ADC (Molecule 3) showed much better cell killing than the anti-Mud1 ADC (Molecule 2) (FIG. 2), which correlated well with previous results that MDA-MB-468 cells have higher expression of EGFR than MUC1 on the cell surface. The cell killing activity of the bispecific anti-MUC1/EGFR ADC (Molecule 1) was similar to that of anti-EGFR ADC (Molecule 3) (FIG. 2), which could be a reflection of the high EGFR expression in this cell line.

On WISH cells, the anti-EGFR ADC (Molecule 3) showed no cell killing activity while the anti-MUC1 ADC (Molecule 2) and the bispecific anti-MUC1/EGFR ADC (Molecule 1) showed potent cell killing activity (FIG. 2), which correlated with previous results that WISH cells have higher expression of MUC1 than EGFR on the cell surface.

On OVCAR-3 cells, anti-EGFR ADC (Molecule 3) and bispecific anti-MUC1/EGFR ADC (Molecule 1) showed more potent cell killing than anti-MUC1 ADC (Molecule 2), but the efficacy (cell killing span) of anti-EGFR ADC (Molecule 3) (65%) was lower than that of anti-MUC1/EGFR ADC (Molecule 1) (88%) and anti-MUC1 ADC (Molecule 2) (89%) (FIG. 2).

No non-specific cell killing activity was observed from the anti-GFP ADC on any of the cells tested, except at the highest concentration on OVCAR-3 cells (FIG. 2). On CHO-k cells, none of the ADCs tested showed any cell killing activity (FIG. 2).

Collectively, these results indicate that, at most concentrations, Molecule 1 specifically kills cancer cells expressing both MUC1 and EGFR.

### Example 5. Effect of Bispecific Anti-MUC1/EGFR ADC on Normal Cells In Vitro

To determine the effect of bispecific anti-MUC1/EGFR ADC on normal (non-cancerous) cells, a cell killing assay was performed with Molecule 1 on HeKn cells (primary normal human epidermal keratinocyte, neonatal) and MCF-10a cells (non-tumorigenic breast epithelial cells). For comparison, the same assay was performed with Molecule 1 on MDA-MB-468 cells (human metastatic breast cancer) and OVCAR-3 cells.

### Methods

MDA-MB-468 cells were cultured in RPMI1640 with stable 300 mg/L L-glutamine and 2.0 g/L NaHCO₃ (Millipore® Sigma, Billerica, MA, USA) supplemented with 10% fetal bovine serum (FBS) (Millipore® Sigma) and 1 mM sodium pyruvate (Gibco®, Thermo Fisher Scientific® or Millipore® Sigma).

OVCAR-3 cells were cultured in RPMI1640 with stable 300 mg/L L-glutamine and 2.0 g/L NaHCO₃ supplemented with 20% FBS, 1 mM sodium pyruvate (Gibco®, Thermo Fisher Scientific®, or Millipore® Sigma), 10 μg/ml insulin (Millipore® Sigma).

Primary human epidermal keratinocytes, neonatal (HeKn) were cultured in basal EpiLife™ medium including human keratinocyte growth supplement (HKGS) on flasks coated with coating matrix (all Gibco®, purchased from Thermo Fisher Scientific®, Waltham, MA, USA).

MCF-10A cells, which are non-tumorigenic breast epithelial cells, were also grown and used for testing. MCF 10A cells were cultured in 1:2 Dulbecco's modified eagle's medium (DMEM) (Millipore® Sigma/Biochrom) with stable glutamine and Ham's F12 (Biochrom) with stable glutamine including 10% horse serum (Gibco®, Thermo Fisher Scientific®) and 20 ng epidermal growth factor (EGF) (Sigma) as well as 500 ng hydrocortisone (Millipore® Sigma).

Molecule 1's cytotoxic effect on cells was measured with a cell proliferation assay. Cell monolayers were washed once with Gibco® D-PBS (Thermo Fisher Scientific®), and cells were detached using ACCUTASE® (Millipore® Sigma) or Gibco® Trypsin/EDTA (#R-001-100) and Trypsin Neutralizer (Gibco® #R-002-100). Viable cells were counted with the automated cell counter LUNA or LUNA-FL™ (Logos Biosystems, Annandale, Virginia, USA) using 0.4% Gibco® trypan blue solution (Thermo Fisher Scientific®). A total of 2,000 cells were seeded in 100 μl cell culture medium (Hekn or MDA-MB-468 cells) or 90 μl cell culture medium (OVCAR-3 or MCF-10 a cells) per well of a 96-well flat bottom cell culture plate (Thermo Fisher Scientific®), which was incubated at 37° C. in a CO₂ incubator overnight. The following day, for Hekn and MDA-MB-468 cells, the medium was replaced by 90 μl fresh cell culture medium with a reduced amount of FBS (3%). The same medium was used to prepare a 10-fold starting concentration of ADC and a respective serial dilution (1:4). For MCF-10a cells or OVCAR-3 cells, there was no medium change. A 10-fold starting concentration of ADC and a respective serial dilution of 1:4 was done using the respective cell culture medium. The respective wells were supplied with 10 μl ADC solution (all treatments were performed in triplicates) and plates were cultured at 37° C. in a CO₂ incubator for 144 hours. Afterwards, 100 μl Cell Titer-Glo® reagent (Promega Corp, Madison, WI, USA) was pipetted in each well, and plates were further processed for cell viability measurement according the manufacturer's instructions. Luminescent signal was measured on a Varioskan® Flash plate reader or Lux plate reader (Thermo Fisher Scientific®). Raw data of relative luminescence units were processed in Microsoft® Excel (version 16.0, Microsoft® Corporation, Redmond, WA, USA) by subtracting the background values (no cell control, only medium) and by converting to % viability (untreated control cells=100%) or % effect (% viability−100%). The dose-response curve and the IC₅₀ value were obtained by data transformation and subsequent data fitting using non-linear regression analysis function (log(inhibitor) versus response-variable slope (three parameters for MDA-MB-468 cells or Hekn cells; four parameters for OVCAR-3 and MCF-10a cells)) in Graph Pad Prism (version 8.2.0) for Windows®, GraphPad software, La Jolla California USA, www.graphpad.com). Data was expressed as % effect vs. ADC concentration [nM] with error bars indicating the SD of the technical triplicates.

### Results

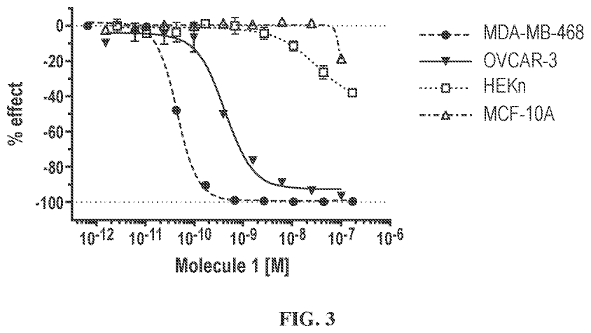

The bispecific anti-MUC1/EGFR ADC (Molecule 1) showed a minimal effect on keratinocyte cell viability and on non-tumor epithelial cells (FIG. 3 and Table 4).

TABLE 4
IC₅₀ and geomean span (% efficacy)of Molecule
1 on tumor and non-tumor cells

| | | | Non-tumor cells | |
|---|---|---|---|---|
| | Tumor cells | | | MCF-10A |
| | MDA-MB-468 | OVCAR-3 | HeKn | mammary |
| | breast cancer | ovarian cancer | keratinocytes | gland |
| MUC1 | + | ++ | (+) | + |
| EGFR | +++ | + | + | (+) |
| IC₅₀ [nM] | 0.05 | 0.2 | 82 | NC |
| Span (%*) | 99 | 96 | 54 | 12 |

n = 3-4;
NC = IC₅₀ not calculable
*at highest concentration tested for Molecule 1

Molecule 1 showed a reduced cell killing efficacy on Hekn (% effect: −54 at highest concentration) and on MCF-10A (% effect: −12 at highest concentration) compared to MDA-MB-468 cells (% effect: −99 at highest concentration). Furthermore, Molecule 1 showed a >1000× fold higher potency on MDA-MB-468 cancer cells (IC₅₀: 0.05 nM) compared to keratinocytes (IC₅₀: 82 nM).

Thus, as shown in FIG. 3, over certain concentration ranges, Molecule 1 effectively kills MDA-MB-468 breast cancer cells while having minimal effects on normal cells.

### Example 6. Internalization of Bispecific Anti-MUC1/EGFR Antibodies by Cancer Cells

### Methods

Internalization of bispecific anti-MUC1/EGFR antibody (Molecule 10), cetuximab (anti-EGFR antibody) (Molecule 9) and H02 IgG1 (anti-MUC1 antibody) (Molecule 11) was evaluated in in vitro on cancer cell lines MDA-MB-468 and OVCAR-3 (both purchased from ATCC, Manassas, VA, USA). MDA-MB-468 cells were cultured in RPMI1640 with stable 300 mg/L L-glutamine and 2.0 g/L NaHCO₃ supplemented with 10% FBS and 1 mM sodium pyruvate (Gibco®, Thermo Fisher Scientific®, or Millipore® Sigma). OVCAR-3 cells were cultured in RPMI1640 with stable 300 mg/L L-glutamine and 2.0 g/L NaHCO₃ supplemented with 20% FBS, 1 mM sodium pyruvate (Gibco®, Thermo Fisher Scientific®, or Millipore® Sigma), 10 μg/ml insulin (Millipore® Sigma). For subculturing, cell monolayers were washed once with Gibco® D-PBS (Thermo Fisher Scientific®), and cells were detached using Accutase®. Viable cells were counted with the automated cell counter LUNA-FL™ using 0.4% Gibco® trypan blue solution. A total of 6,000 MDA-MB-468 cells or 10000 OVCAR-3 cells were seeded in 90 μl cell culture medium per well of a 96-well plate (Corning®, NY, USA). The plates were incubated overnight in the incubator at 37° C. and 5% CO₂. The following day, nuclear staining was performed using Hoechst 33342 (Thermo Fisher Scientific®) at a final concentration of 0.5 μg/ml. Ten microliters of a 10× stock solution prepared in PBS was added per well. The plate was incubated for 30 min in the incubator at 37° C. and 5% CO₂. The medium was removed afterwards, and the wells were supplied with 90 μl fresh cell culture medium. Each antibody used for testing was incubated with Zenon™ pHrodo™ iFL Red Human IgG labeling reagent (Thermo Fisher Scientific®) for 5 min in the dark (protein:dye molar ratio used: 1:3). The antibody-pHrodo™ mixture with a final concentration of 100 nM antibody was added per well (technical duplicates were performed), followed by 25 min incubation at 37° C. and 5% CO₂ to initiate internalization. Measurement was performed 30 min, 150 min, 390 min, 24 h and 48 h after addition of the antibody-pHrodo™ mixture. Cells were imaged with the confocal quantitative image cytometer CQ1 (Yokogawa® Electric Corporation, Tokyo, Japan) using the following imaging conditions: 20× objective lens, 405 nM laser (Hoechst), 561 nM laser (pHrodo™ iFL Red). Five z-stacked images per well were collected (z-stack range: 20 μm, slice: 1 μm) and the CQ1 Software (version 1.04.02.04, Yokogawa) was used to determine internalization into acidic cell compartments by quantifying the mean intensity of pHrodo™ red signal, derived from a specific area around the nuclei. For data processing, Microsoft® Excel (version 16.0, Microsoft® Corporation, Redmond, WA, USA) and Graph Pad Prism (version 8.2.0 for Windows®, GraphPad software, La Jolla California USA) were used. Internalization data are displayed as mean intensity versus time [h] with error bars indicating the Standard Deviation (SD) of the duplicates.

### Results

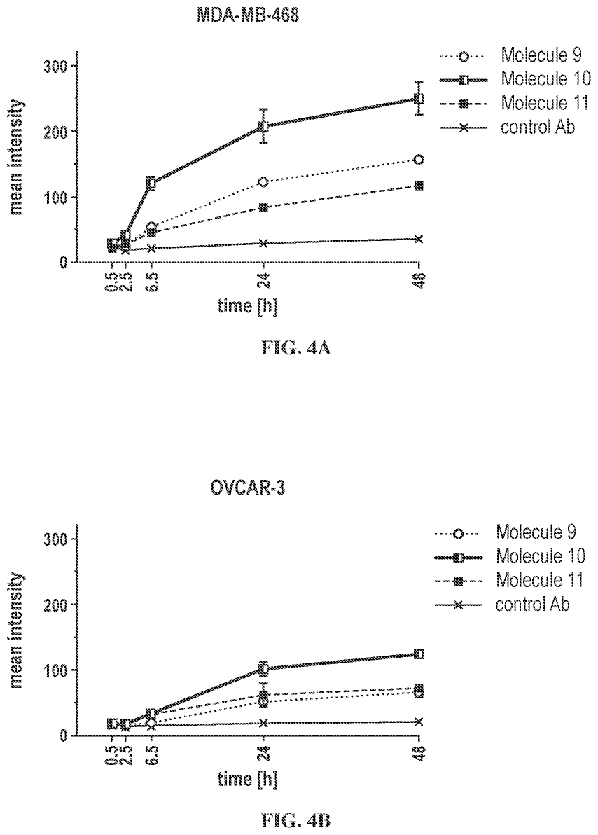

For internalization studies, bispecific anti-MUC1/EGFR antibody (Molecule 10) as well as the monospecific control antibodies H02 IgG1 (anti-MUC1 antibody) (Molecule 11) and cetuximab (anti-EGFR antibody) (Molecule 9) were labeled with the Zenon™ pHrodo™ iFL Red dye (Thermo Fisher Scientific®), which turns fluorescent in the acidic environment. Internalization of the pHrodo™-iFL-labeled antibodies to acidic cell compartments was evaluated over time in OVCAR-3 and MDA-MB-468 cells by live cell imaging using the CQ1 device and by measuring the mean fluorescence intensity of the pH-sensitive dye (see FIGS. 4A and 4B).

On MDA-MB-468 and OVCAR-3 cells, the bispecific anti-MUC1/EGFR antibody H02/hC225 (Molecule 10) showed rapid internalization and trafficking to acidic compartments. Molecule 10 continued to be internalized during the 48 h of incubation time, as determined by increased mean fluorescence intensity over time (FIGS. 4A and 4B). In both cell lines, the mean fluorescence intensity obtained for Molecule 10 was much stronger compared to the one obtained for the monospecific control antibodies H02 IgG1 (Molecule 11) and cetuximab (Molecule 9).

### Example 7. Bispecific Anti-MUC1/EGFR ADC Kills Wild Type and Mutant EGFR Cancer Cells In Vitro

To determine the effect of bispecific anti-MUC1/EGFR ADC on cancer cells having different EGFR mutational status, cell killing assays were performed using Molecule 1 on EGFR wild type (wt) cells, EGFR exon deletion mutant cells, and EGFR double substitution mutant cells.

### Methods

NSCLC cells NCI-H292 (EGFR wild type (wt)), HCC827 (EGFR del E746-A750), and NCI-H1975 (EGFR L858R/T790M) were all purchased from ATCC. NCI-H292 and NCI-H1975 were cultured in RPMI1640 media with stable L-glutamine (Millipore® Sigma), 10% FBS (Millipore® Sigma) and 1 mM sodium pyruvate (Gibco®, Thermo Fisher Scientific®, or Millipore® Sigma). HCC827 cells were cultured in RPMI1640 media with stable 2 mM L-glutamine, 2.5 g/L D-(+)-glucose solution (Millipore® Sigma), 10 mM HEPES (Millipore® Sigma), 10% FBS, and 1 mM sodium pyruvate (Gibco®, Thermo Fisher Scientific®, or Millipore® Sigma).

The expression levels of MUC1 or EGFR on NSCLC cells were evaluated by FACS using H02 IgG1 (anti-MUC1 antibody) or cetuximab (anti-EGFR antibody), respectively, and by calculating the median fluorescence intensity (MFI) ratio (MFI target-specific antibody/MFI isotype). The expression levels were determined to be + for a MFI ratio up to 100, ++ for a MFI ratio >100, or +++ for a MFI ratio >200. According to this, the expression levels for the NSCLC cells were defined to be MUC1+/EGFR++(NCI-H292 cells), MUC1+/EGFR+++(HCC827 cells) or MUC1+/EGFR+(NCI-H1975).

For cytotoxicity testing, cell monolayers were washed once with Gibco® D-PBS (Thermo Fisher Scientific®) and detached from the cell culture flask using Accutase® (Millipore® Sigma). Viable cells were counted with the automated cell counter LUNA-FL™ (Logos Biosystems) using 0.4% Gibco® trypan blue solution (Thermo Fisher Scientific®). A total of 625 cells (NCI-H292), 1250 cells (NCI-H1975) or 3000 cells (HCC827) were plated in 90 μl cell culture medium per well of a 96-well black/clear flat bottom TC-treated imaging microplate (Corning®) and cultured overnight. A ten-fold starting concentration of ADCs or compounds and a respective serial dilution (1:4) were prepared in cell culture medium briefly before use. Wells were supplied with 10 μl ADC or compound solution. Treatment was performed in technical triplicates. The plates were subsequently incubated for 144 h at 37° C. and 5% CO₂. Untreated control cells for ADCs or EGFR tyrosine kinase inhibitors (TKIs) received a corresponding amount of dilution media or dimethylsulfoxide (DMSO; Millipore Sigma), respectively. For subsequent cell viability measurement, 100 μl Cell Titer-Glo® reagent (Promega™ Corp, Madison, WI, USA) was pipetted in each well, and plates were further processed according the manufacturer's instructions. Luminescent signal was measured on a Varioskan® Flash plate reader (Thermo Fisher Scientific®). Raw data of relative luminescence units were processed in Microsoft® Excel (version 16.0, Microsoft® Corporation, Redmond, WA, USA) by subtracting the background values (no-cell control, only medium) and by calculating the % viability (untreated control cells=100%) or % effect (% viability−100%). Dose-response curve and IC₅₀ values were obtained by data transformation and subsequent data fitting using a non-linear regression analysis function (log(inhibitor) vs. response-variable slope (four parameters)) in Graph Pad Prism (version 8.2.0 for Windows®, GraphPad software, La Jolla California USA). Data was expressed as % effect vs. dose of compound concentration [M] with error bars indicating the SD of the technical triplicates.

### Results

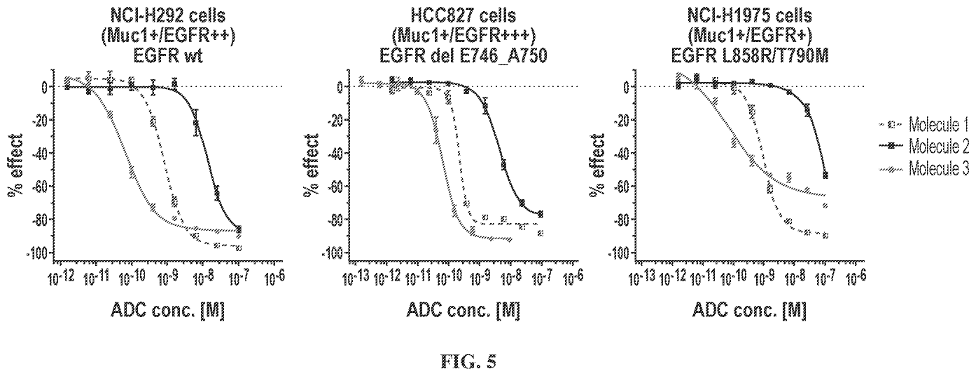

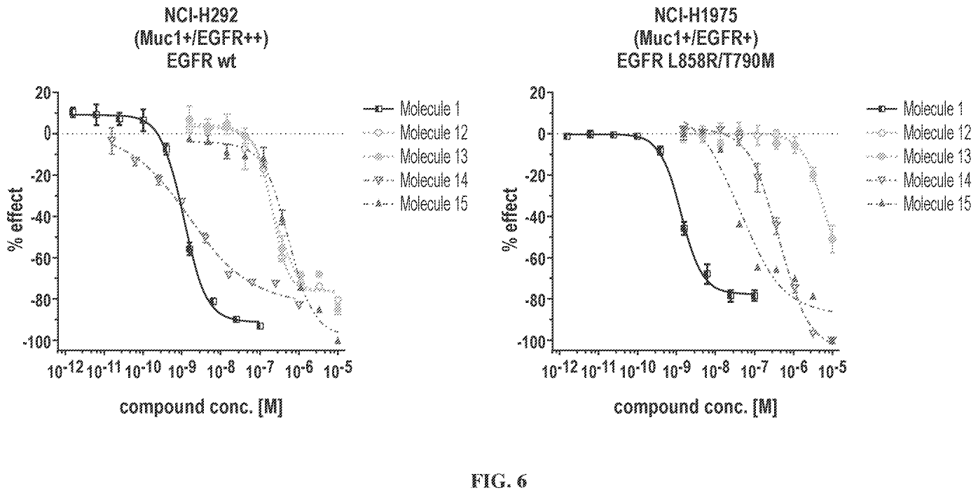

The cell killing activity of the bispecific anti-MUC1/EGFR ADC (Molecule 1) was evaluated on NSCLC cells with different EGFR mutational status (NCI-H292: EGFR wt; HCC827: EGFR del E746_A750; and NCI-H1975: EGFR L858R/T790M). Monospecific ADCs anti-MUC1 ADC (Molecule 2) and anti-EGFR ADC (Molecule 3) were used as control molecules (FIG. 5, Table 5). For NCI-H292 and NCI-H1975 cells, additional cell killing assays were performed with Molecule 1 and the EGFR TKIs erlotinib (Molecule 12), gefitinib (Molecule 13), afatinib (Molecule 14) and osimertinib (Molecule 15) (FIG. 6, Table 6). The potency of the ADCs or EGFR TKIs were determined in several individual experiments as geomean IC₅₀ [nM] as the mean concentration from the obtained cell killing curves at which 50% of the maximum inhibition of cell viability was observed. In addition, geomean span (%) (the mean percentage of cells that were killed relative to the untreated control cells at the highest test concentration used) was determined. The geomean span represents the efficacies of ADCs or compounds in cell viability assays.

As shown in FIG. 5, Molecule 1 showed high efficacy and potency on NSCLC cells in the cell viability assay, independent of their MUC1 and EGFR expression levels and their EGFR mutation status (EGFR wt, EGFR L858R/T790M, or EGFR del E746_A750).

For NCI-H292 (EGFR wt) cells, anti-EGFR ADC (Molecule 3) showed higher potency compared to the anti-MUC1 ADC (Molecule 2) (FIG. 5, Table 5). A higher sensitivity to Molecule 3 than to Molecule 2 may be a result of the higher expression level of EGFR and relatively low expression level of MUC1 in these cells. According to these results, the cell killing activity of the bispecific anti-MUC1/EGFR ADC (Molecule 1) is in between those of the monospecific ADCs (Molecule 2 and 3). However, Molecule 1 showed slightly better efficacy than each of the monospecific ADCs, which may indicate a synergistic action.

TABLE 5
Geomean IC₅₀ values [nM] and geomean span (% efficacy) (n = 1-4) for FIG. 5

| | | | NCI-H292 | | HCC827 | | NCI-H1975 | |
|---|---|---|---|---|---|---|---|---|
| | | | IC₅₀ | Span | IC₅₀ | Span | IC₅₀ | Span |
| Molecule No. | Sample | DAR | [nM] | (%) | [nM] | (%) | [nM] | (%) |
| Molecule 1 | bispecific anti- | 4.0 | 1.1 | 95 | 0.23 | 88 | 0.83 | 90 |
| | MUC1/EGFR ADC | | | | | | | |
| Molecule 2 | anti-MUC1 ADC | 3.8 | 20 | 59 | 4.5 | 77 | 310* | 54 |
| Molecule 3 | anti-EGFR ADC | 3.7^{#1} | 0.17 | 89 | 0.06 | 93 | 0.04 | 75 |
| | | 3.9^{#2} | | | | | | |

DAR = drug-antibody ratio
*estimated
^{#1}for NCI-H292 and NCI-H1975;
^{#2}for HCC-827

Bispecific anti-MUC1/EGFR ADC (Molecule 1) showed a cell killing activity on EGFR mutant cells HCC827 (EGFR del E746_A750) in the subnanomolar range with a killing span (%) that is comparable to the monospecific anti-EGFR ADC (Molecule 3) (FIG. 5, Table 5). In these cells with high expression levels of EGFR, both Molecule 3 and Molecule 1 inhibited cell viability at comparable potency.

On the EGFR double mutant (L858R/T790M) NCI-H1975 cells, which expresses both antigens at a lower level, the anti-EGFR ADC (Molecule 3) showed superior potency compared to the anti-MUC1 ADC (Molecule 2) and a higher activity than the bispecific anti-MUC1/EGFR ADC (Molecule 1) with regard to cell viability inhibition (FIG. 5, Table 5). However, Molecule 1 showed a better cell killing efficacy of 90% compared to Molecule 2 and Molecule 3, having killing spans of 54% and 75%, respectively. These results may indicate a synergistic activity of the bispecific ADC over the monospecific ADC variants on cells with lower MUC1 and EGFR expression levels.

FIG. 6 and Table 6 show the results from experiments performed using small molecule EGFR TKIs. The results from Molecule 1 are also shown for comparison.

TABLE 6
Geomean IC₅₀ values [nM] and geomean span (% efficacy) at
highest test concentration (n = 1-4) for FIG. 6

| | | | NCI-H292 | | NCI-H1975 | |
|---|---|---|---|---|---|---|
| | | | IC₅₀ | Span | IC₅₀ | Span |
| Molecule No. | Sample | DAR | [nM] | (%) | [nM] | (%) |
| Molecule 1 | bispecific anti- | 4.0 | 0.4 | 96 | 1.0 | 87 |
| | MUC1/EGFR ADC | | | | | |
| Molecule 12 | erlotinib | NA | 280 | 82 | NC | NC |
| Molecule 13 | gefitinib | NA | 230 | 87 | NC | NC |
| Molecule 14 | afatinib | NA | 1.9 | 91 | 590 | 100 |
| Molecule 15 | osimertinib | NA | 570 | 100 | 43 | 100 |

NC = Not calculable due to incomplete dilution curve

The sensitivities of selected NSCLC cells NCI-H292 (EGFR wt) and NCI-H1975 (EGFR L858R/T790M) to different EGFR TKIs was consistent with results described in the literature (FIG. 6, Table 4; reference: Hirano et al., In vitro modeling to determine mutation specificity of EGFR tyrosine kinase inhibitors against clinically relevant EGFR mutants in non-small-cell lung cancer, Oncotarget 2015, 6, 38789-38803). Consistent with results described by Hirano et al., afatinib (Molecule 14) inhibited wild-type EGFR most effectively compared to the other TKI inhibitors (Molecules 12, 13, 15) whereas osimertinib (Molecule 15), an EGFR TKI selective for targeting T790M resistance mutation, showed highest cell killing activity in NCI-H1975 cells (FIG. 6, Table 6).

In summary, bispecific anti-MUC1/EGFR ADC (Molecule 1) demonstrated potency in the sub-nanomolar range against both wild type and mutant EGFR cells, which are characterized by varying expression levels for MUC1 and EGFR.

### Example 8. Pharmacokinetic Properties of Bispecific Anti-MUC1/EGFR ADC in Rodents

The non-compartmental pharmacokinetic (PK) parameters of Molecule 1 was evaluated in non-tumor bearing female CB17 SCID mice and Sprague-Dawley rats.

### Methods

In mice, a single 5 mg/kg IV bolus was administered, sampled at different time-points, and pooled from different animals (non-repeated measures). In rats, a single 5 mg/kg dose by IV bolus was administered via an indwelling jugular vein catheter, and blood samples were collected at different time-points using repeated measures design.

### Results

A summary of the results is presented in Table 7.

TABLE 7
Pharmacokinetic parameters of Molecule 1 in Rodents

| Parameters | Units | CB17 SCID | Rat |
|---|---|---|---|
| Dose | mg/kg | 5 | 5 |
| Study length | Days | 21 | 21 |
| T_{1/2} | Days | 12.1 | 9.5 |
| C₀ | μg/mL | 107 | 147 |
| Cmax ± SE | μg/mL | 104 ± 5 | 144 ± 3 |
| AUC₍₀₋ₐₗₗ₎ ± | day* μg/mL | 474 ± 10 | 636 ± 19 |
| SEM | | | |
| AUC_{(0-∞)} | day* μg/mL | 698 | 803 |
| CL | mL/day/kg | 7.16 | 6.2 |
| Vss | mL/kg | 126 | 82.1 |

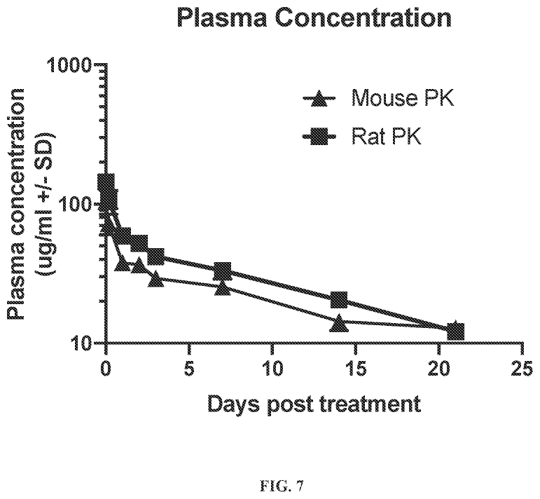

The elimination half-life (T_{1/2}) was determined from a regression analysis of the log-linear plot of the concentration-time curves. The PK parameters including T_{1/2}, CL, and Vss of Molecule 1 were comparable in mice and rats (FIG. 7). In addition, Molecule 1 exhibited rodent PK profiles that appear similar those of other FDA-approved monoclonal IgG antibodies.

### Example 9. Dose Response Efficacy Study of Bispecific Anti-MUC1/EGFR ADC in a Cervical Cancer (WISH) Xenograft Model

The dose-response relationship of the bispecific anti-MUC1/EGFR ADC Molecule 1 was evaluated in WISH tumors, a human cervical cell line (HeLa contaminant) which expresses the highest endogenous levels of MUC1 (+++) relative to all other cell lines tested, and low endogenous levels of EGFR (+) in two independent studies.

### Methods

Female athymic nude mice with established WISH tumors (˜150 mm³) were treated with a single intravenous (IV) injection of Molecule 1 at doses ranging from 0.1 mg/kg to 1.5 mg/kg (Study 1) or 1.25 mg/kg to 5 mg/kg (Study 2).

### Results

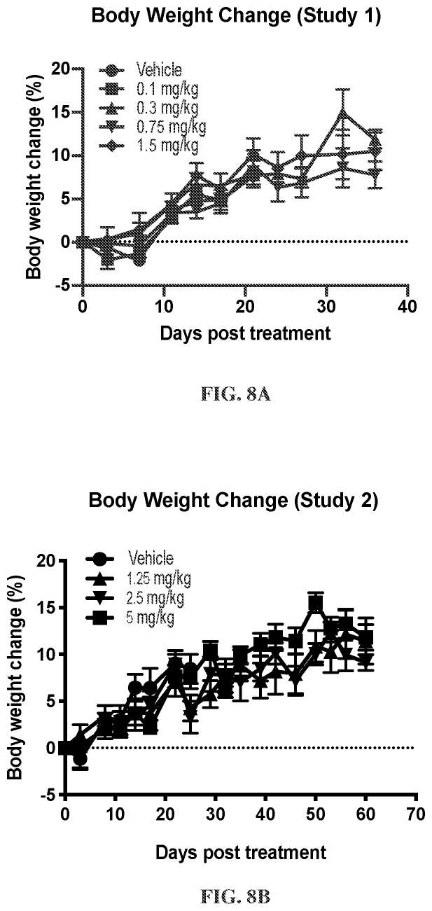

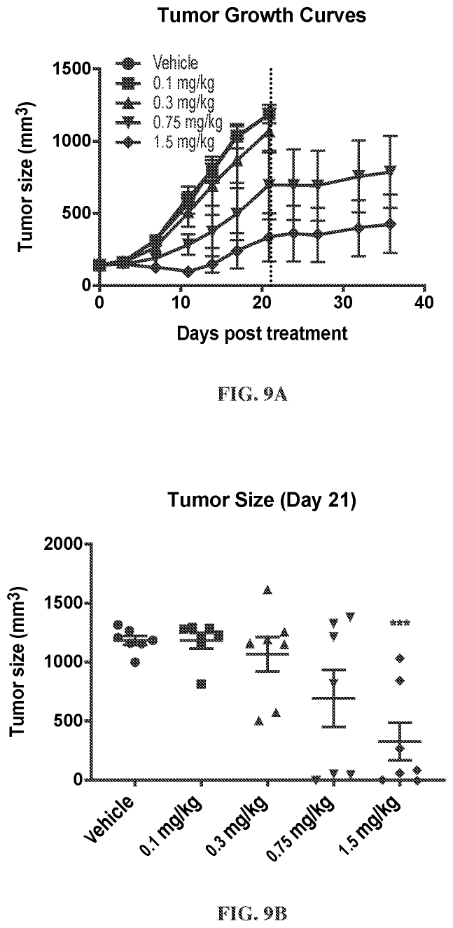

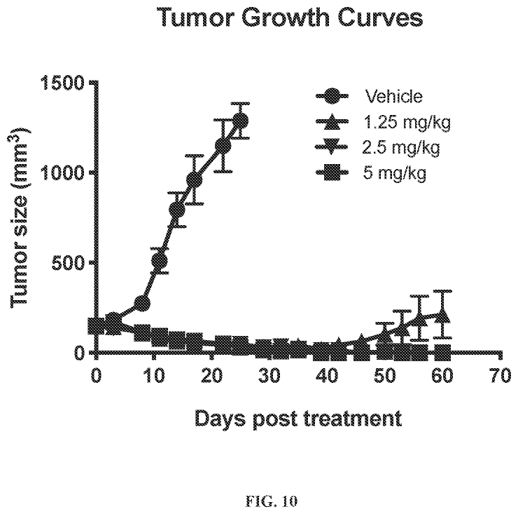

In both studies, treatment was well tolerated with no toxicity and normal weight gain observed (FIGS. 8A and 8B). The effects of treatment on WISH tumor growth and the individual tumor sizes on the day the vehicle control treated tumors reached the study endpoint (>1,200 mm³) are illustrated in FIGS. 9A, 9B, and 10. In Study 1, Molecule 1 administered at 0.1, 0.3, 0.75 and 1.5 mg/kg demonstrated dose dependent anti-tumor activity. The lowest doses tested, 0.1 and 0.3 mg/kg, showed poor efficacy at 0% and 12% tumor growth inhibition (TGI), respectively (FIGS. 9A and 9B). Moderate activity (47% TGI) was observed with 0.75 mg/kg Molecule 1, while 1.5 mg/kg elicited significant activity at 81% TGI (p=0.0009) compared to vehicle control based on statistical analysis of tumor size on day 21 (FIG. 8B). The highest dose of Molecule 1 (1.5 mg/kg) initially induced tumor regression with re-growth observed approximately 10 days after treatment (FIG. 9A). In Study 2, a single dose of Molecule 1 at 1.25, 2.5 and 5 mg/kg demonstrated robust and significant efficacy as evidenced by induction of tumor regression at all doses (FIG. 10). At the end of the study, complete responses were observed in greater than 50% (4 out of 7) of the animals treated with the lowest dose and 100% (7 out of 7) of animals that received 2.5 and 5 mg/kg of Molecule 1.

In conclusion, results from both studies independently demonstrated potent anti-tumor activity of Molecule 1 resulting in significant efficacy as well as tumor regression in WISH tumors. The minimum efficacious dose (MED), defined as the lowest dose to induce a >20% decrease in tumor volume from baseline (for any time point post treatment initiation), was consistent in both studies and determined at to be approximately 1.5 mg/kg of Molecule 1 in the WISH tumor model.

### Example 10. Dose Response Efficacy Study of Bispecific Anti-MUC1/EGFR ADC in an Ovarian Cancer (OVCAR-3) Xenograft Model

The dose-response relationship of the bispecific anti-MUC1/EGFR ADC Molecule 1 was evaluated in OVCAR-3 tumors, a human ovarian adenocarcinoma which expresses low endogenous levels of both MUC1 (++) and EGFR (+).

### Methods

Female CB17 SCID (severe combined immunodeficient, C.B-17-IcrHSD-Prkdc^{scid}) mice with established OVCAR-3 tumors (˜100 mm³) were treated with a single IV injection of Molecule 1 at doses ranging from 2.5 mg/kg to 10 mg/kg.

### Results

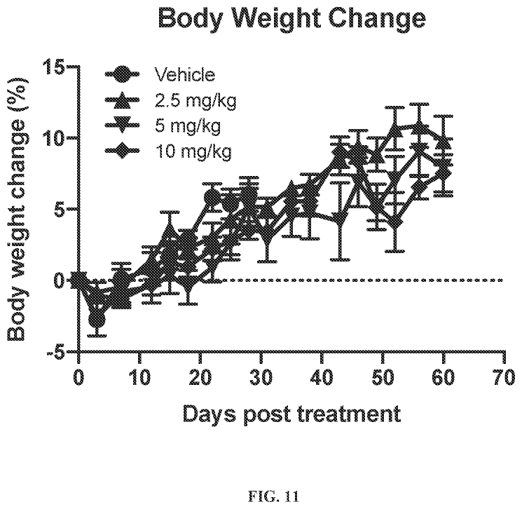

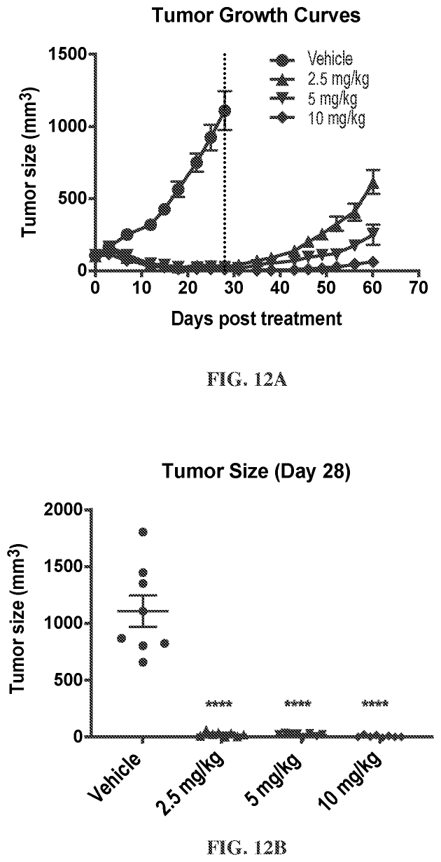

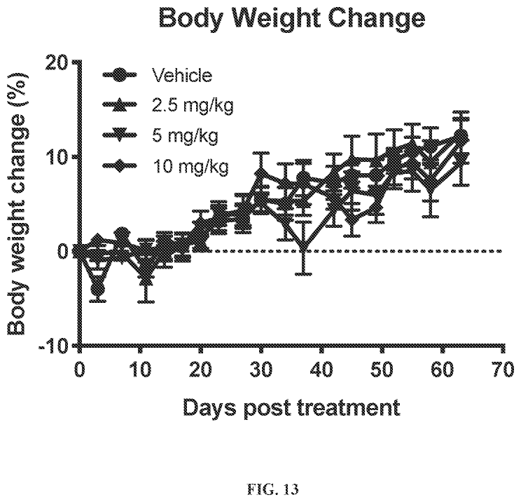

Treatment was well tolerated, with no toxicity or clinical signs noted, as well as significant (p≤0.05) mean body weight gain between 3.10% and 6.04% of initial weight at all doses tested (FIG. 11). Molecule 1 at 2.5, 5 and 10 mg/kg induced tumor regression (>100% TGI) and suppressed growth until approximately day 31 post treatment (FIG. 12A). Analysis of tumor size on day 28 showed that Molecule 1 was significantly (p<0.0001) efficacious compared to the vehicle control (FIG. 12B). In conclusion, Molecule 1 demonstrated potent efficacy in OVCAR-3 tumors.

### Example 11. Dose Response Efficacy Study of Bispecific Anti-MUC1/EGFR ADC in a Breast Cancer (MDA-MB-468) Xenograft Model

The dose-response relationship of the bispecific anti-MUC1/EGFR ADC Molecule 1 was evaluated in MDA-MB-468 tumors, a human breast metastatic adenocarcinoma model which expresses lower levels of MUC1 expression (+) relative to the high EGFR level (+++).

### Methods

Female SCID Beige mice with established MDA-MB-468 tumors (˜130 mm³) were treated with a single intravenous injection of Molecule 1 at doses ranging from 2.5 mg/kg to 10 mg/kg.

### Results

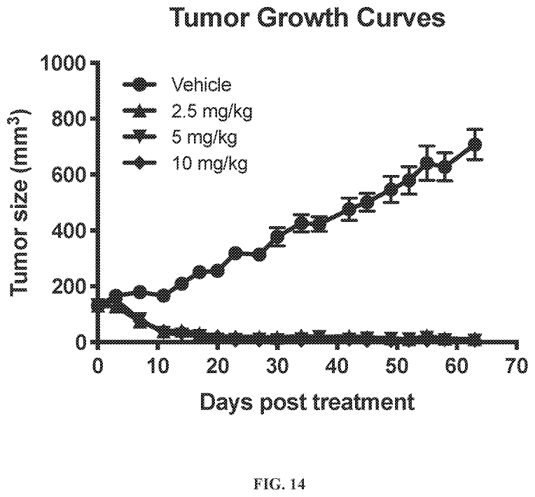

Treatment was well tolerated, with no toxicity and normal weight gain observed (FIG. 12). All doses of Molecule 1 (2.5, 5 and 10 mg/kg) induced significant anti-tumor activity, achieving complete tumor regression and suppressing growth until the end of study on day 63 post treatment (FIG. 14). In conclusion, Molecule 1 exhibited potent activity leading to tumor regression and prolonged duration of response in the MDA-MB-468 model.

### Example 12. Dose Response Efficacy Study of Bispecific Anti-MUC1/EGFR ADC in a Non-Small Cell Lung Cancer (NSCLC) Patient-Derived Xenograft Model

The dose-response relationship of the bispecific anti-MUC1/EGFR ADC Molecule 1 was evaluated in the NSCLC Patient-derived xenograft (PDX) model LUX089. This PDX model expresses both MUC1 and EGFR.

### Methods

Female nude mice (Nu/Nu, Vital River Laboratory Animal Technology Co. Ltd., Beijing, China) with established LUX089 tumors (˜150 mm³) were treated with a single IV injection of Molecule 1 at doses ranging from 2 mg/kg to 10 mg/kg.

### Results

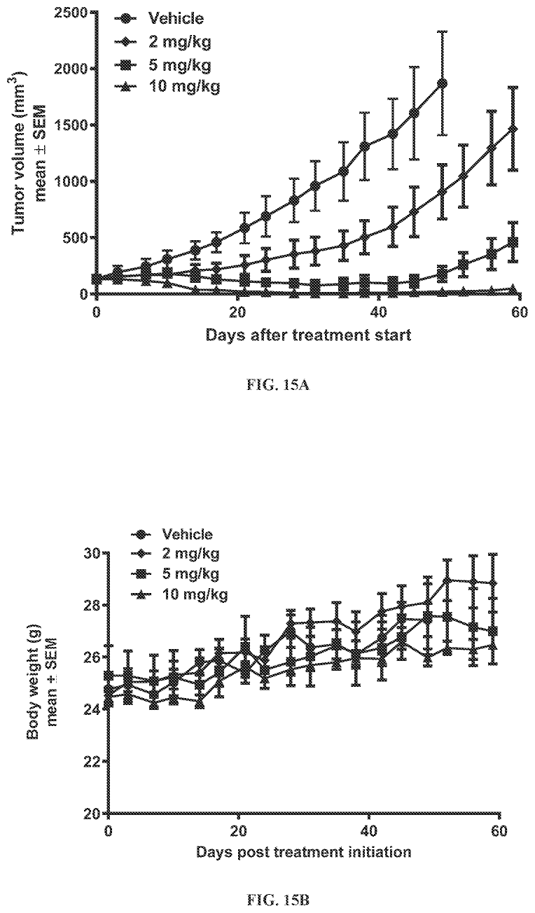

Treatment was well tolerated, with no toxicity or clinical signs noted and no weight loss observed (FIG. 15B). Molecule 1 administered once at day 0 at 2, 5 and 10 mg/kg showed a dose-dependent tumor growth inhibition, with complete regression in the 10 mg/kg dose group (FIG. 15A). In three of the five animals treated in the 10 mg/kg dose group, no tumor was measurable up to day 60, when the experiment was finished. The 2 and 5 mg/kg doses caused a TGI of 69% and 24% regression (p<0.001), respectively, at Day 38, when the vehicle tumor reached an average volume of 1300 mm³. In conclusion, Molecule 1 demonstrated potent efficacy in the PDX model LUX089.

### Example 13. Comparison Between Bispecific Anti-MUC1/EGFR ADC and Monospecific ADCs in a Non-Small Cell Lung Cancer (NSCLC) Patient-Derived Xenograft Model

The efficacy of the bispecific anti-MUC1/EGFR ADC Molecule 1 in comparison to monospecific anti-EGFR and anti-MUC1 ADCs was evaluated in three different NSCLC patient-derived xenograft (PDX) models at the same dose. All three PDX models express both MUC1 and EGFR.

### Methods

Female nude (Nu/Nu, Vital River Laboratory Animal Technology Co. Ltd., Beijing, China) mice with established LUX089, LUX019 and LUX003 tumors (˜150 mm³) were treated with a single IV injection of Molecule 1 and the monospecific ADCs at a dose of 5 mg/kg.

### Results

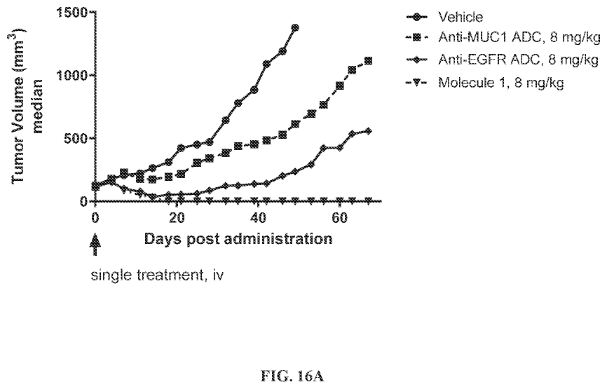

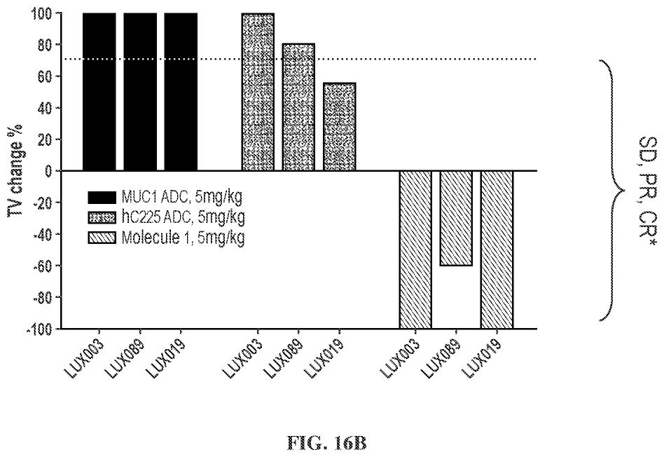

In all three PDX models, Molecule 1 showed the strongest tumor growth inhibition, with complete regression in model LUX003 and LUX019. In LUX089, the treatment caused partial regression. The second most efficacious treatment, the anti-EGFR ADC, resulted in tumor stasis in the model LUX019. The anti-MUC1 ADC did not cause tumor shrinkage in the three tested PDX models at 5 mg/kg single treatment. (FIG. 16). In conclusion, Molecule 1 showed the strongest anti-tumor efficacy in the three tested NSCLC PDX models.

### Example 14. Efficacy of Bispecific Anti-MUC1/EGFR ADC in Patient-Derived Xenograft Models of Different Cancer Indications

The efficacy of the bispecific anti-MUC1/EGFR ADC Molecule 1 was tested in PDX models from different cancer indications. Indications were selected based on known expression levels of MUC1 and EGFR.

### Methods

Female nude (Nu/Nu) mice with PDX models from NSCLC, gastric cancer, esophageal cancer, ovarian cancer, breast cancer, head and neck cancer, cervical cancer, and mesothelioma were treated with a single IV injection of Molecule 1 at a dose of 8 mg/kg. Efficacy was assessed as Progressive disease (PD), Stable disease (SD); Partial regression (PR) or complete regression (CR) at the day of best response (if tumor response delayed)/when the vehicle group tumor volume (median) reached 1000 mm³ using the following criteria: tumor volume change >73%, <73% and >−66%, ≤−66%, correspond to PD, SD, PR and tumors not measurable correspond to CR.

### Results

Strong anti-tumor responses (partial or complete regressions) were observed in all tested indications. Responses were seen in models expressing varying levels of MUC1 or EGFR. In Table 8, NSCLC PDX models which express high EGFR and MUC1 expression levels (based on immunohistochemistry scoring >15 using the haloscore software) were marked with an asterisk. NSCLC PDX models with EGFR mutations (LUPF049: EGFR19del (748-753); LUPF104: EGFR19 del (746-750), T790M, and C797S) were marked with a hashtag. In conclusion, Molecule 1 showed a broad applicability in several cancer indications expressing varying levels of MUC1 and EGFR.

TABLE 8
Treatment response in PDX models from different indications

| Indication | Model | Response |
|---|---|---|
| NSCLC (adeno¹ or squamous² | LUX010² | PD |
| cell carcinoma) | LUX019²* | CR |
| | LUX034² | PD |
| | LUX106¹ | PD |
| | LUX003²* | SD |
| | LUX101² | PD |
| | LUX110² | PD |
| | CTC160128²* | CR |
| | CTC15008¹* | CR |
| | LUPF049¹# | CR |
| | LUPF104¹# | CR |
| Esophageal squamous | ESX008 | SD |
| cell carcinoma | ESX019 | CR |
| | ESX005 | PR |
| | ESX076 | PD |
| | ESX030 | CR |
| | ESX006 | CR |
| | EC002 | CR |
| | ESPF160344 | CR |
| | ESPF160802 | CR |
| | ESPF160845 | CR |
| | ESPF160845 | CR |
| | OES13497 | CR |
| | ESPF161825 | CR |
| | ESPF161498LY | CR |
| Gastric cancer | GAX001 | PD |
| | GAX066 | SD |
| | GAX031 | CR |
| Breast cancer (TNBC) | CTG-1019 | CR |
| | CTG-1018 | CR |
| | HBCx-8 | PR |
| | HBCx-17 | PR |
| | CTG-1018 | CR |
| | CTG-1019 | CR |
| | HBCx-6 | CR |
| | BCX-017-LOP | CR |
| | HBCx-28 | CR |
| Head and Neck squamous | HNX005 | CR |
| cell carcinoma | HN12656 | SD |
| | HN11218 | CR |
| | HN11269B | CR |
| | HN11857A | CR |
| | HN13194 | CR |
| | HN14755 | CR |
| | HN10632 | CR |
| | HN10309 | CR |
| Ovarian cancer | OVPF040 | PR |
| | OVPF167 | PD |
| | OVPF169 | SD |
| | OVPF174 | SD |
| | OVPF027 | PR |
| | OVPF042 | CR |
| | OVPF041 | CR |
| | OVX046 | SD |
| Cervical Cancer | CEPF002 | SD |
| | CEPF160042 | PR |
| | CEPF012 | CR |
| | CEPF101 | CR |
| | CEPF161330 | CR |
| | CEX009 | CR |
| | CER14951 | CR |
| Mesothelioma | PNX334 | SD |
| | PNX411 | SD |
| | PNX392E | CR |

PD = progressive disease
SD = stable disease
PR = partial regression
CR = complete regression

### Example 15. Efficacy of Bispecific Anti-MUC1/EGFR ADC in Patient-Derived Xenograft Models Using Different Treatment Schedules

The efficacy of the bispecific anti-MUC1/EGFR ADC Molecule 1 was tested in an NSCLC PDX model using different schedules.

### Methods

Female nude (Nu/Nu, Vital River Laboratory Animal Technology Co. Ltd., Beijing, China) mice with established patient-derived NSCLC tumors (˜150 mm³) were treated with a single IV injection of 8 mg/kg Molecule 1, two IV injections of 4 mg/kg Molecule 1 one week or two week apart, or four IV injections of 2 mg/kg Molecule 1 weekly.

### Results

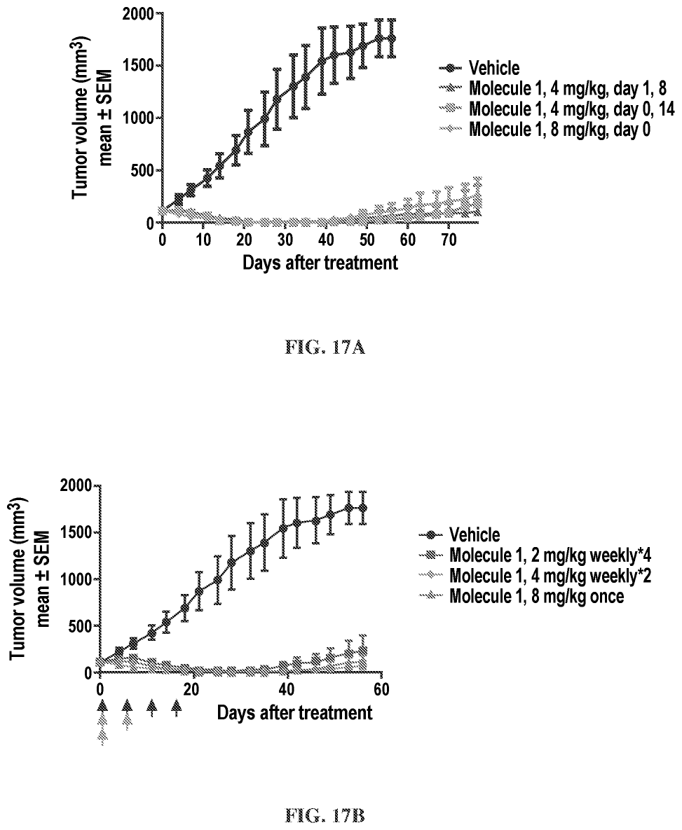

In all schedules, using the same total dose of 8 mg/kg induced strong and durable tumor growth inhibition (FIGS. 17A and 17B). In conclusion, Molecule 1 showed the strong anti-tumor efficacy using different treatment regimens.

### Example 16. Efficacy of Bispecific Anti-MUC1/EGFR ADC in Patient-Derived Xenograft Models from NSCLC, Esophageal Cancer, and Head and Neck Squamous Cell Carcinoma

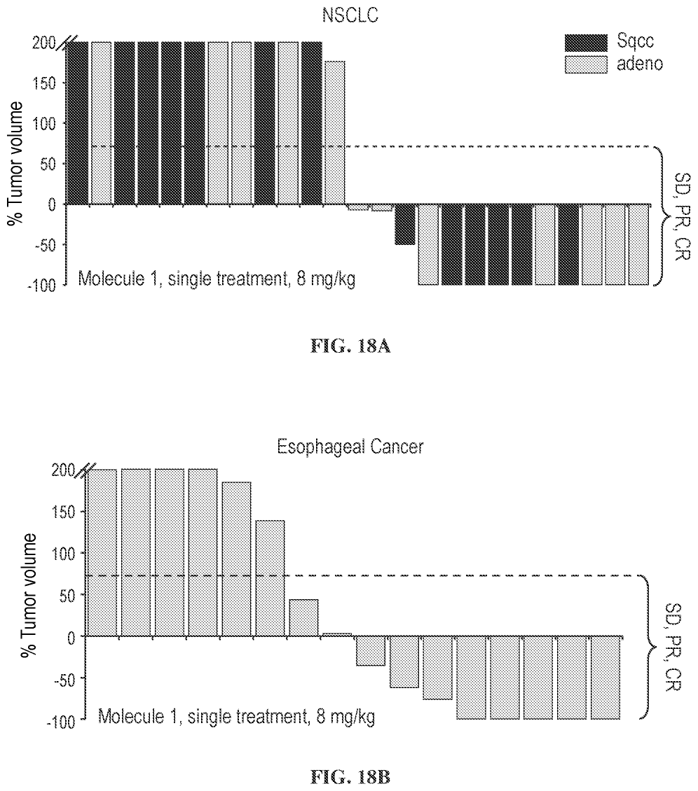

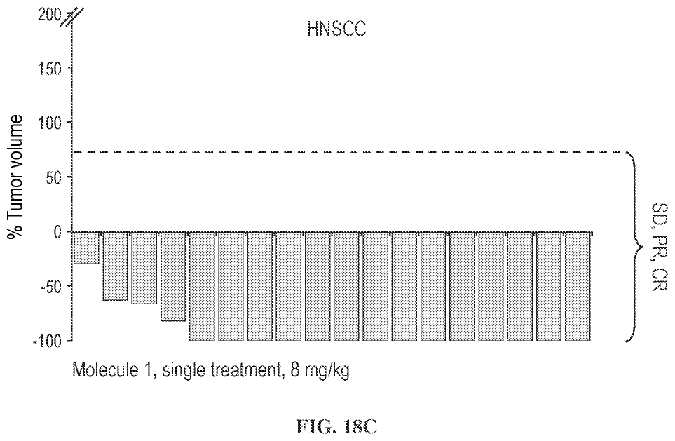

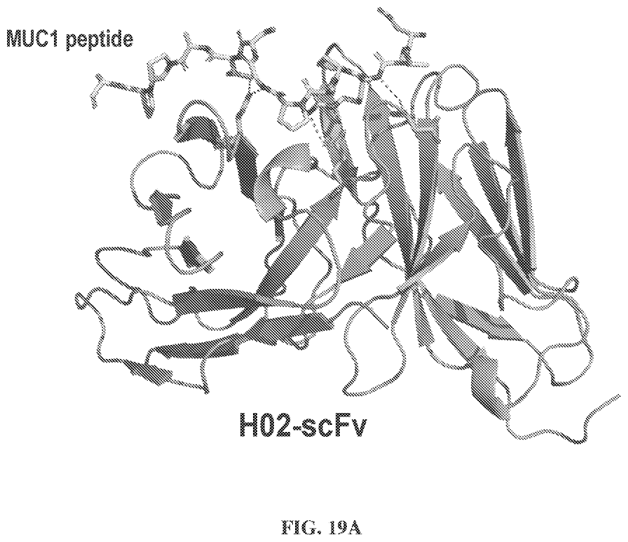

The efficacy of a single 8 mg/kg dose of Molecule 1 was tested in a variety of patient-derived xenograft models from NSCLC, esophageal cancer, and head and neck squamous cell carcinoma. As shown in FIGS. 18A, 18B, and 18C, a substantial fraction of patient-derived xenografts from NSCLC, esophageal cancers, and head and neck squamous cell carcinomas exhibited complete remission after a single dose. Tumor response was associated with target expression.

### Example 17. Epitope Mapping of Anti-MUC1 Antibodies

To determine the minimal binding epitope of the anti-MUC1 arm of Molecule 1, PEPperMAP® Epitope Mappings of human anti-MUC1 antibodies HT186-D11 and H02, and were performed against human MUC1 peptide APDTRPAPGSTAPPAHGVTSAPDTRPAPGSTAPPAHGVTS (SEQ ID NO:22) translated into linear 15, 12 and 10 amino acid peptides with peptide-peptide overlaps of 14, 11 and 9 amino acids as well as against sequence truncations of 15 amino acid peptides APDTRPAPGSTAPPA (SEQ ID NO:23), PAPGSTAPPAHGVTS (SEQ ID NO:24), TAPPAHGVTSAPDTR (SEQ ID NO:25) and HGVTSAPDTRPAPGS (SEQ ID NO:26). The resulting peptide microarrays were incubated with the antibody samples at a concentration of 1 μg/ml in incubation buffer followed by staining with the secondary and control antibodies as well as read-out with a LI-COR Odyssey Imaging System. Quantification of spot intensities and peptide annotation were done with PepSlide® Analyzer.

Pre-staining of a peptide microarray copy did not highlight any background interaction of the secondary or control antibodies with the peptide variants of the wild type peptide that could interfere with the main assays. In contrast, incubation with the antibody samples resulted in very similar and very clear IgG response profiles. Antibody HT186-D11 showed the strongest response against peptides with the minimal consensus motif TRPAP (SEQ ID NO:27). The same minimal consensus motif was recognized by antibody H02, albeit at moderate spot intensities. A strong response was also found with antibody H02 with interactions with peptides with the minimal consensus motif DTRPAP (SEQ ID NO:28). Removal of the C-terminal proline or the N-terminal threonine resulted in a significant decrease of spot intensities and hence antibody binding.

### Example 18. Kinetic Interaction Analysis of Molecule 1 Against Human and Cynomolgus Monkey EGFR

To assess binding affinities for EGFR, Molecule 1 (anti-MUC1/EGFR ADC; see Example 3) and Molecule 10 (unconjugated anti-MUC1/EGFR; see Example 2) were immobilized on biosensor tips. Association and dissociation of soluble analytes (human EGFR or cynomolgus monkey (Macaca fascicularis) EGFR; “cyno EGFR”) were measured as the interference shift in nm that directly resulted from protein binding to the tips of the biosensors. The data was processed to obtain kₒₙ, k_{dis} and K_{D} values using a 1:1 interaction model and global curve fitting.

Results are shown in Tables 9A and 9B. The dissociation constants (K_{D}) of Molecule 1 against human and cyno EGFR were in the low single-digit nM range (1.5 nM). The kinetic binding constant of Molecule 10 was very similar, at approximately 1.4 nM.

Thus, Molecule 1 (anti-MUC1/EGFR ADC) binds to EGFR with similar kinetics as unconjugated anti-MUC1/EGFR (Molecule 10). These results demonstrate that conjugation of the hemiasterlin derivative to make Molecule 1 (see Example 3) does not impact binding to EGFR.

TABLE 9A
Results including calculated K_{D}, kₒₙ, and k_{dis} values of Molecule 1 and
Molecule 10 against human EGFR.
Analyte: human EGFR

| | | kₒₙ (1/Ms) | k_{dis} (1/s) |
|---|---|---|---|
| Sample | K_{D} (nM) | (error; % error/Kₒₙ) | (error; % error/K_{dis}) |
| Molecule 1 | 1.465 | 6.24E+05 | 9.14E−04 |
| | | (2.47E+03; 0.396%) | (2.27E−06; 0.249%) |
| Molecule 10 | 1.444 | 6.73E+05 | 9.72E−04 |
| | | (3.11E+03; 0.462%) | (2.71E−06; 0.279%) |

TABLE 9B
Results including calculated K_{D}, kₒₙ, and k_{dis} values of Molecule 1 and
Molecule 10 against cyno EGFR.
Analyte: cyno EGFR-His

| | | kₒₙ (1/Ms) | k_{dis} (1/s) |
|---|---|---|---|
| Sample | K_{D} (nM) | (error; % error/Kₒₙ) | (error; % error/K_{dis}) |
| Molecule 1 | 3.5 | 8.38E+05 | 2.94E−03 |
| | | (6.51E+03; 0.777%) | (2.67E−05; 0.907%) |
| Molecule 10 | 3.4 | 9.16E+05 | 3.11E−03 |
| | | (1.12E+04; 1.226%) | (4.23E−05; 1.359%) |

### Example 19. Kinetic Interaction Analysis of Molecule 1 Against Human and Cynomolgus Monkey MUC1

To assess binding affinities for MUC1, Molecule 1 (anti-MUC1/EGFR ADC; see Example 3) and Molecule 10 (unconjugated anti-MUC1/EGFR; see Example 2) were immobilized on a C1 series S sensor chip via covalent coupling on primary amines using the respective amine coupling kit. Association and dissociation of 1000 nM of the analytes (cyno (Macaca fascicularis) MUC1 peptide and human MUC1 peptide VHH fusion) were measured for 180 sec each.

Read-outs were measured responses directly resulting from protein binding to surfaces of the sensor chips. The data was processed to obtain kₒₙ, k_{dis} and K_{D} values using a heterogeneous interaction model and global curve fitting.

Results are shown in Table 10. The measured dissociation constants (K_{D}) against human MUC1 peptide (as an N-terminal fusion to a camelid VHH) were 21.5 nM for Molecule 1 and 47.2 nM for Molecule 10. The curve shape for each of these molecules indicated a heterogeneous binding mode. This second interaction appears to be significantly weaker for all tested molecules. No interaction could be measured with the cyno MUC1 peptide

Thus, Molecule 1 (anti-MUC1/EGFR ADC) binds to human MUC1 with similar kinetics as unconjugated anti-MUC1/EGFR (Molecule 10). These results demonstrate that conjugation of the hemiasterlin derivative to make Molecule 1 (see Example 3) does not significantly impact binding to MUC1.

The lack of binding to the cyno MUC1 peptide may be due to species specific differences in the amino acid sequence. As described in Example 17, the anti-MUC1 binding arm of Molecule 1 was determined to have a minimal binding epitope that comprises the amino acid sequence TRPAP (SEQ ID NO:27). A sequence alignment of MUC1 of different species shows that this minimal epitope is not present in cyno and rodent MUC1.

TABLE 10
Results including calculated K_{D}, kₒₙ, and k_{dis} values
of Molecule 1 and Molecule 10 against human MUC1.
Analyte: human MUC1-VHH fusion

| | kₐ₁ | k_{d1} | K_{D1} | kₐ₂ | k_{d2} | K_{D2} |
|---|---|---|---|---|---|---|
| Sample | (1/Ms) | (1/s) | (M) | (1/Ms) | (1/s) | (M) |
| Molecule 1 | 4.55E+04 | 9.78E−04 | 2.15E−08 | 9.89E+05 | 3.09E−01 | 3.13E−07 |
| Molecule 10 | 2.85E+04 | 1.34E−03 | 4.72E−08 | 3.59E+05 | 4.80E−01 | 1.34E−06 |

### Example 20. Binding Mode to MUC1

To obtain further insight to the binding mode of H02-scFv, the MUC1-binding arm of Molecule 1, the crystal structure of a complex between H02-scFv and a fragment of human MUC1 immunodominant core peptide (APDTRPAPGSTAPPA; SEQ ID NO:23) was solved.

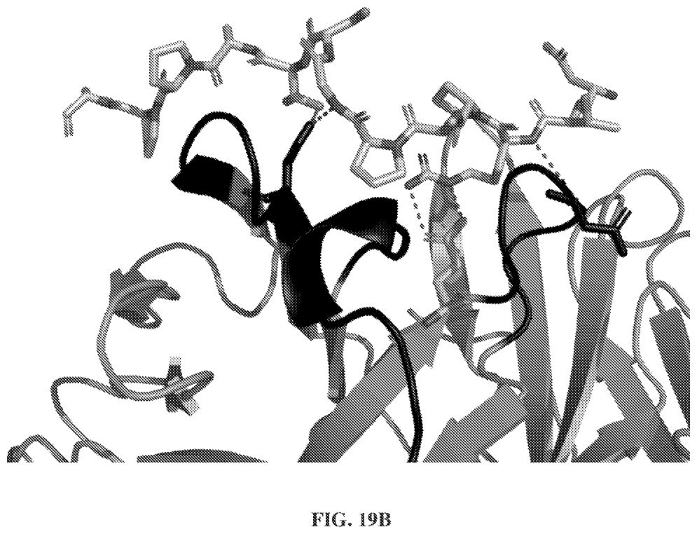

Prior to crystallization, H02-scFv was incubated with 10× molar excess of the MUC1 peptide on ice for 30 minutes and subsequently concentrated to 22 mg/ml in 25 mM HEPES, 150 mM NaCl, pH 7.4 buffer. Crystals were grown at 277 K using hanging drop vapor diffusion technique by mixing 1.0 μl protein solution with 1.0 μl reservoir solution (0.1 M Tris, 0.2 M MgCl₂, 28% w/v PEG4000, pH 8.5). The overall structure of the complex is shown in FIG. 19A.

Within the crystal structure, the MUC1 peptide chain is well defined from Asp 3 to Ala 15 in the electron density map (2Fo-Fc), as shown in FIG. 19A. Arg 5 [MUC1]'s side chain guanidinium forms a bidentate salt bridge with the carboxylate group of Glu 99 [H02-scFv], whereas Arg 5 [MUC1]'s main chain nitrogen forms a hydrogen bond with the main chain carbonyl oxygen of Asp 103 [H02-scFv]. Two more hydrogen bonds are observed: 1) between Gly 9 [MUC1]'s main chain nitrogen and the carboxylate of Asp 52 [H02-scFv] and 2) between Thr 11 Oγ [MUC1] and Asp 52 [H02-scFv]. The interaction between the MUC1 peptide and H02-scFv is further stabilized by van der Waals contacts, especially by Pro 8 [MUC1] binding into the cavity formed by the CDR1 Thr 30-His32 patch of H02-scFv and by the CDR2 Asp 52-Val54 patch of H02-scFv. The rest of the contacts between MUC1 peptide and H02-scFv are mediated by water molecules.

### EQUIVALENTS

The entire disclosure of each of the patent documents and scientific articles referred to herein is incorporated by reference for all purposes.

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

SEQUENCES
(anti-MUC1 scFvFc (AG SEED))
SEQ ID NO: 1
MQMQLVQSEAELKKPGASVKVSCKASGYSFTSHFMHWVRQAPGQGLEWMGWIDPVTGGTK
YAQNFQGWVTMTRDTSIRTAYLELSRLRSDDTAMYYCAREARADRGQFDKWGQGTLVTVA
SGGGGSGGGGSGGGGSQSVLTQPPSVSVAPGKTARITCGGNNIGSKSVHWYQQKPGQAPA
LVIYYGSNRPSGIPERFSGSNSGNTATLTISRVEAGDEADYYCQVWDSSSDWVFGGGTKL
TVLKPKSSDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPI
EKTISKAKGQPFRPEVHLLPPSREEMTKNQVSLTCLARGFYPKDIAVEWESNGQPENNYK
TTPSRQEPSQGTTTFAVTSKLTVDKSRWQQGNVFSCSVMHEALHNHYTQKTISLSPGK
(anti-EGFR Fab(heavy)Fc (GA SEED))
SEQ ID NO: 2
NTPFTSRVTITSDKSTSTAYMELSSLRSEDTAVYYCARALTYYDYEFAYWGQGTLVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPPSEE
LALNELVTLTCLVKGFYPSDIAVEWLQGSQELPREKYLTWAPVLDSDGSFFLYSILRVAA
EDWKKGDTFSCSVMHEALHNHYTQKSLDRSPGK
(anti-EGFR Fab(light chain))
SEQ ID NO: 3
MDIQMTQSPSSLSASVGDRVTITCRASQSIGTNIHWYQQKPGKAPKLLIKYASESISGVP
SRFSGSGYGTDFTLTISSLQPEDVATYYCQQNNNWPTTFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
(CDRH1 motif for HT186-D11 and 1993 series antibodies)
SEQ ID NO: 4
GYX₁FX₂X₃X₄X₅MH
wherein
X1 is S (serine) or P (proline),
X2 is T (threonine) or N (asparagine),
X3 is G (glycine), D (aspartic acid), or S (serine),
X4 is H (histidine) or N (asparagine), and
X5 is Y (tyrosine) or F (phenylalanine).
(CDRH2 motif for HT186-D11 and 1993 series antibodies)
SEQ ID NO: 5
WIDPVTGX₁TX₂YAQX₃FQG
wherein
X1 is G (glycine) or E (glutamic acid),
X2 is K (lysine) or R (arginine), and
X3 is N (asparagine) or D (aspartic acid).
(CDRH3 motif for HT186-D11 and 1993 series antibodies)
SEQ ID NO: 6
EX₁X₂X₃X₄RGQFDK
wherein
X1 is V (valine) or A (alanine),
X2 is T (threonine) or R (arginine),
X3 is G (glycine) or A (alanine), and
X4 is D (aspartic acid) or S (serine).
(CDRL1 for HT186-D11 and 1993 series antibodies)
SEQ ID NO: 7
GGNNIGSKSVH
(CDRL2 for HT186-D11 and 1993 series antibodies)
SEQ ID NO: 8
YGSNRPS
(CDRL3 for HT186-D11 and 1993 series antibodies)
SEQ ID NO: 9
QVWDSSSDWV
(VNTR peptide of MUC1)
SEQ ID NO: 10
APDTRPAPGSTAPPAC
(anti-MUC1 scFvFc (AG SEED) with non-natural amino acids
(e.g., pAMF) introduced at sites indicated by *)
SEQ ID NO: 11
MQMQLVQSEAELKKPGASVKVSCKASGYSFTSHFMHWVRQAPGQGLEWMGWIDPVTGGTK
YAQNFQGWVTMTRDTSIRTAYLELSRLRSDDTAMYYCAREARADRGQFDKWGQGTLVTVA
SGGGGSGGGGSGGGGSQSVLTQPPSVSVAPGKTARITCGGNNIGSKSVHWYQQKPGQAPA
LVIYYGSNRPSGIPERFSGSNSGNTATLTISRVEAGDEADYYCQVWDSSSDWVFGGGTKL
TVLKPKSSDKTHTCPPCPAPELLGGPSV*LFPPKPKDTLMISRTPEVTCVVVDVSHEDPE
VKFNWYVDGVEVHNAKTKPREEQYNSTYRVVSVLTVLHQDWLNGKEYKCKVSNKALPAPI
EKTISKAKGQPFRPEVHLLPPSREEMTKNQVSLTCLARGFYPKDIAVEWESNGQPENNYK
TTPSRQEPSQGTTT*AVTSKLTVDKSRWQQGNVFSCSVMHEALHNHYTQKTISLSPGK
((anti-EGFR Fab(heavy)Fc (GA SEED) with non-natural
amino acids (e.g., pAMF) introduced at sites indicated by *)
SEQ ID NO: 12
NTPFTSRVTITSDKSTSTAYMELSSLRSEDTAVYYCARALTYYDYEFAYWGQGTLVTVSS
ASTKGPSVFPLAPSSKSTSGGTAALGCLVKDYFPEPVTVSWNSGALTSGVHTFPAVLQSS
GL*SLSSVVTVPSSSLGTQTYICNVNHKPSNTKVDKRVEPKSCDKTHTCPPCPAPELLGG
PSV*LFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPPSEE
LALNELVTLTCLVKGFYPSDIAVEWLQGSQELPREKYLTWAPVLDSDGSFFLYSILRVAA
EDWKKGDTFSCSVMHEALHNHYTQKSLDRSPGK
(CDRH1 for hC225)
SEQ ID NO: 13
GFSLTNYG
(CDRH2 for hC225)
SEQ ID NO: 14
IWSGGNT
(CDRH3 for hC225)
SEQ ID NO: 15
ARALTYYDYEFAY
(CDRL1 for hC225)
SEQ ID NO: 16
QSIGTN
(CDRL2 for hC225)
SEQ ID NO: 17
YASE
(CDRL3 for hC225)
SEQ ID NO: 18
QQNNNWPTT
(CH1 within hC225 heavy chain)
SEQ ID NO: 19
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKRV
(CH1 within hC225 heavy chain with non-natural amino acid
(e.g., pAMF) introduced at site indicated by *)
SEQ ID NO: 20
ASTKGPSVFPLAPSSKSTSGGTAALGCLVKDYFPEPVTVSWNSGALTSGVHTFPAVLQSS
GL*SLSSVVTVPSSSLGTQTYICNVNHKPSNTKVDKRV
(CL1 within hC225 light chain)
SEQ ID NO: 21
RTVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
(peptide from human MUC1)
SEQ ID NO: 22
APDTRPAPGSTAPPAHGVTSAPDTRPAPGSTAPPAHGVTS
(human MUC1 truncated peptide)
SEQ ID NO: 23
APDTRPAPGSTAPPA
(human MUC1 truncated peptide)
SEQ ID NO: 24
PAPGSTAPPAHGVTS
(human MUC1 truncated peptide)
SEQ ID NO: 25
TAPPAHGVTSAPDTR
(human MUC1 truncated peptide)
SEQ ID NO: 26
HGVTSAPDTRPAPGS
(human MUC1 minimal consensus motif)
SEQ ID NO: 27
TRPAP
(human MUC1 minimal consensus motif)
SEQ ID NO: 28
DTRPAP
(CDRH1 for HT186-D11-Kabat)
SEQ ID NO: 29
GHYMH
(CDRH2 for HT186-D11-Kabat)
SEQ ID NO: 30
WIDPVTGGTKYAQNFQG
(CDRH3 for HT186-D11-Kabat)
SEQ ID NO: 31
EVTGDRGQFDK
(CDRH1 for HT186-D11-Chothia)
SEQ ID NO: 32
GYSFTGH
(CDRH2 for HT186-D11-Chothia)
SEQ ID NO: 33
DPVTGG
(CDRH3 for HT186-D11-Chothia)
SEQ ID NO: 34
EVTGDRGQFDK
(CDRH1 for 1993-H02-Kabat)
SEQ ID NO: 35
SHFMH
(CDRH2 for 1993-H02-Kabat)
SEQ ID NO: 36
WIDPVTGGTKYAQNFQG
(CDRH3 for 1993-H02-Kabat)
SEQ ID NO: 37
EARADRGQFDK
(CDRH1 for 1993-H02-Chothia)
SEQ ID NO: 38
GYSFTSH
(CDRH2 for 1993-H02-Chothia)
SEQ ID NO: 39
DPVTGG
(CDRH3 for 1993-H02-Chothia)
SEQ ID NO: 40
EARADRGQFDK
(VH for HT186-D11)
SEQ ID NO: 41
QMQLVQSEAELKKPGASVKVSCKASGYSFTGHYMHWVRQAPGQGLEWMGWIDPVTGGTKYAQ
NFQGWVTMTRDTSIRTAYLELSRLRSDDTAMYYCAREVTGDRGQFDKWGQGTLVTVAS
(VH for 1993-H02)
SEQ ID NO: 42
QMQLVQSEAELKKPGASVKVSCKASGYSFTSHFMHWVRQAPGQGLEWMGWIDPVTGGTKYAQ
NFQGWVTMTRDTSIRTAYLELSRLRSDDTAMYYCAREARADRGQFDKWGQGTLVTVAS
(VL for HT186-D11 and for 1993-H02)
SEQ ID NO: 43
QSVLTQPPSVSVAPGKTARITCGGNNIGSKSVHWYQQKPGQAPALVIYYGSNRPSGIPERFS
GSNSGNTATLTISRVEAGDEADYYCQVWDSSSDWVFGGGTKLTVL
(VH for 1993-E03)
SEQ ID NO: 44
QMQLVQSEAELKKPGASVKVSCKASGYSFNDHFMHWVRQAPGQGLEWMGWIDPVTGGTKYAQ
NFQGWVTMTRDTSIRTAYLELSRLRSDDTAMYYCAREARADRGQFDKWGQGTLVTVAS
(VH for 1993-H08)
SEQ ID NO: 45
QMQLVQSEAELKKPGASVKVSCKASGYSFTGHYMHWVRQAPGQGLEWMGWIDPVTGETKYAQ
NFQGWVTMTRDTSIRTAYLELSRLRSDDTAMYYCAREARADRGQFDKWGQGTLVTVAS
(VH for 1993-G09)
SEQ ID NO: 46
QMQLVQSEAELKKPGASVKVSCKASGYSFTDHYMHWVRQAPGQGLEWMGWIDPVTGETKYAQ
DFQGWVTMTRDTSIRTAYLELSRLRSDDTAMYYCAREARADRGQFDKWGQGTLVTVAS
(VH for 1993-E04)
SEQ ID NO: 47
QMQLVQSEAELKKPGASVKVSCKASGYSFTGNYMHWVRQAPGQGPEWMGWIDPVTGETKYAQ
NFQGWVTMTRDTSIRTAYLELSRLRSDDTAMYYCAREARASRGQFDKWGQGTLVTVAS
(VH for 1993-D01)
SEQ ID NO: 48
QMQLVQSEAELKKPGASVKVSCKASGYPFTGHYMHWVRQAPGQGPEWMGWIDPVTGETRYAQ
NFQGWVTMTRDTSIRTAYLELSRLRSDDTAMYYCAREARADRGQFDKWGQGTLVTVAS

1. An immunoconjugate comprising:
(a) a bispecific antibody that binds to EGFR and MUC1, the bispecific antibody comprising:
(i) a first polypeptide comprising a first engineered Fc domain and a single-chain Fv (scFv), wherein the scFv binds MUC1,
(ii) a second polypeptide comprising a second engineered Fc domain and a heavy chain of a Fab fragment, and
(iii) a third polypeptide comprising a light chain of the Fab fragment;
wherein the second and third polypeptide chains together define an Fab fragment that binds EGFR,
wherein the first polypeptide and the second polypeptide are covalently linked by one or more disulfide bonds formed between the first engineered Fc domain and the second engineered Fc domain;
wherein the second polypeptide and the third polypeptide are covalently linked by one or more disulfide bonds formed between the heavy chain of the second polypeptide and the light chain of the third polypeptide; and
wherein the first polypeptide and the second polypeptides each comprise at least one non-natural amino acid residue; and
(b) a plurality of hemiasterlin moieties, wherein each hemiasterlin moiety is independently conjugated via a linker to one of the non-natural amino acid residues of the first polypeptide or the second polypeptide.

2. The immunoconjugate of claim 1, wherein the plurality of hemiasterlin moieties comprises four hemiasterlin moieties.

3-5. (canceled)

6. The immunoconjugate of claim 1, wherein the first engineered Fe domain comprises two non-natural amino acid residues.

7. (canceled)

8. The immunoconjugate of claim 6, wherein the first engineered Fc domain comprises non-natural amino acid residues at heavy chain positions F241 and F404 according to the EU index.

9. The immunoconjugate of claim 1, wherein the second engineered Fc domain comprises a non-natural amino acid residue.

10. (canceled)

11. The immunoconjugate of claim 9, wherein the second engineered Fc domain comprises a non-natural amino acid residue at heavy chain position F241 according to the EU index.

12. The immunoconjugate of claim 1, wherein the Fab fragment comprises a non-natural amino acid residue.

13-14. (canceled)

15. The immunoconjugate of claim 12, wherein the Fab fragment comprises a non-natural amino acid residue at heavy chain position Y180 according to the EU index.

16. The immunoconjugate of claim 1, wherein each of the at least one non-natural amino acid residues is selected from the group consisting of p-acetyl-L-phenylalanine, O-methyl-L-tyrosine, 3-methyl-phenylalanine, O-4-allyl-L-tyrosine, 4-propyl-L-tyrosine, fluorinated phenylalanine, isopropyl-L-phenylalanine, p-azido-L-phenylalanine, p-acyl-L-phenylalanine, p-benzoyl-L-phenylalanine, p-iodophenylalanine, p-bromophenylalanine, p-amino-L-phenylalanine, isopropyl-L-phenylalanine, p-propargyloxyphenylalanine, and p-azidomethyl-L-phenylalanine.

17-18. (canceled)

19. The immunoconjugate of claim 1, wherein the first polypeptide comprises complementarity-determining regions (CDRs):
CDR-L1 comprising the amino acid sequence set forth in SEQ ID NO:7;
CDR-L2 comprising the amino acid sequence set forth in SEQ ID NO:8; and
CDR-L3 comprising the amino acid sequence set forth in SEQ ID NO:9.

20. The immunoconjugate of claim 19, wherein the first polypeptide comprises complementarity-determining regions (CDRs):
CDR-H1 comprising the amino acid sequence set forth in SEQ ID NO:4,
CDR-H2 comprising the amino acid sequence set forth in SEQ ID NO:5, and
CDR-H3 comprising the amino acid sequence set forth in SEQ ID NO:6.

21. The immunoconjugate of claim 19, wherein the first polypeptide comprises complementarity-determining regions (CDRs):
(a) (i) CDR-H1 comprising the amino acid sequence set forth in SEQ ID NO:29,
(ii) CDR-H2 comprising the amino acid sequence set forth in SEQ ID NO:30, and
(iii) CDR-H3 comprising the amino acid sequence set forth in SEQ ID NO:31; or
(b) (i) CDR-H1 comprising the amino acid sequence set forth in SEQ ID NO:32,
(ii) CDR-H2 comprising the amino acid sequence set forth in SEQ ID NO:33, and
(iii) CDR-H3 comprising the amino acid sequence set forth in SEQ ID NO:34.

22. (canceled)

23. The immunoconjugate of claim 19, wherein the first polypeptide comprises complementarity-determining regions (CDRs):
(a) (i) CDR-H1 comprising the amino acid sequence set forth in SEQ ID NO:35,
(ii) CDR-H2 comprising the amino acid sequence set forth in SEQ ID NO:36, and
(iii) CDR-H3 comprising the amino acid sequence set forth in SEQ ID NO:37; or
(b) (i) CDR-H1 comprising the amino acid sequence set forth in SEQ ID NO:38,
(ii) CDR-H2 comprising the amino acid sequence set forth in SEQ ID NO:39, and
(iii) CDR-H3 comprising the amino acid sequence set forth in SEQ ID NO:40.

24. (canceled)

25. The immunoconjugate of claim 1, wherein the second polypeptide comprises complementarity-determining regions (CDRs):
CDR-H1 comprising the amino acid sequence set forth in SEQ ID NO:13,
CDR-H2 comprising the amin acid sequence set forth in SEQ ID NO:14, and
CDR-H3 comprising the amino acid sequence set forth in SEQ ID NO:15.

26. The immunoconjugate of claim 1, wherein the third polypeptide comprises complementarity-determining regions (CDRs):
CDR-L1 comprising the amino acid sequence set forth in SEQ ID NO:16,
CDR-L2 comprising the amino acid sequence set forth in SEQ ID NO:17, and
CDR-L3 comprising the amino acid sequence set forth in SEQ ID NO:18.

27-32. (canceled)

33. The immunoconjugate of claim 1, wherein the linker is a cleavable linker.

34. (canceled)

35. The immunoconjugate of claim 1, wherein the hemiasterlin moiety is a hemiasterlin derivative.

36. (canceled)

37. The immunoconjugate of claim 35, wherein the immunoconjugate comprises the following structure: 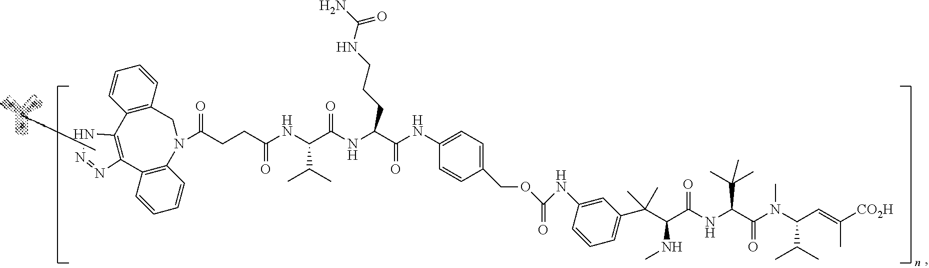
wherein n is 4.

38. An immunoconjugate comprising:
(a) a bispecific antibody that binds to EGFR and MUC1, the bispecific antibody comprising:
(i) a first polypeptide comprising a first engineered Fe domain and a single-chain Fv fragment (scFv), wherein the scFv binds to MUC1, the first polypeptide chain comprising the amino acid sequence of SEQ ID NO:11 that comprises a non-natural amino acid residue at heavy chain positions F241 and F404 according to the EU index,
(ii) a second polypeptide comprising a second engineered Fc domain and a heavy chain of a Fab fragment, the second polypeptide comprising the amino acid sequence of SEQ ID NO:12 that comprises a non-natural amino acid residue at positions Y180 and F241 according to the EU index, and
(iii) a third polypeptide comprising a light chain of the Fab fragment, the third polypeptide comprising the amino acid sequence of SEQ ID NO:3;
wherein the second and third polypeptide chains together define a Fab fragment that binds EGFR,
wherein the first polypeptide and the second polypeptide are covalently linked by one or more disulfide bonds formed between the first engineered Fc domain and the second engineered Fc domain, and
wherein the second polypeptide and the third polypeptide are covalently linked by one or more disulfide bonds formed between the heavy chain of the second polypeptide and the light chain of the third polypeptide; and
(b) a plurality of 3-aminophenyl hemiasterlin moieties, each independently conjugated via a cleavable valine-citrulline-p-aminobenzylalcohol linker to one of the non-natural amino acid residues.

39. The immunoconjugate of claim 38, wherein the immunoconjugate comprises four 3-aminophenyl hemiasterlin moieties.

40. (canceled)

41. A pharmaceutical composition comprising the immunoconjugate of claim 1 and a pharmaceutically acceptable carrier.

42. A method of treating cancer in a mammalian subject in need thereof, the method comprising the step of:
administering a therapeutically effective amount of the immunoconjugate of claim 1 to the subject.

43-83. (canceled)
